(19) United States
(12) Patent Application Publication
(10) Pub. No.: US 2021/0378571 A1
(43) Pub. Date: Dec. 9, 2021

(54) COMPOSITE BIOELECTRODES

(71) Applicant: Facebook Technologies, LLC, Menlo Park, CA (US)

(72) Inventors: Raymond King, Woodinville, WA (US); Brendan Patrick Fiynn, Renton, WA (US); Shawn Reese, Renton, WA (US); Jonathan Shea Robinson, Kirkland, WA (US)

(21) Appl. No.: 17/330,375

(22) Filed: May 25, 2021

Related U.S. Application Data
(60) Provisional application No. 63/035,420, filed on Jun. 5, 2020.

(51) Int. Cl. A61B 5/25; A61B 5/332; A61B 5/00

(52) U.S. Cl. CPC A61B 5/25; A61B 2562/227; A61B 5/6823; A61B 5/332

(57) ABSTRACT

A computing device may include (1) biosignal-acquiring circuitry that captures a biosignal from a user's body and (2) one or more composite bioelectrodes electrically coupled to the biosignal-acquiring circuitry. The one or more composite bioelectrodes may include (1) a circuitry-interfacing side with a mechanical or electrical property having a first predetermined configuration and (2) a user-interfacing side with the mechanical or electrical property having a second predetermined configuration. Various other composite bioelectrodes, systems, and methods are also disclosed.

### CROSS REFERENCE TO RELATED APPLICATION

This application claims the benefit of U.S. Provisional Application No. 63/035,420, filed 5 Jun. 2020, the disclosure of which is incorporated, in its entirety, by this reference.

### BRIEF DESCRIPTION OF THE DRAWINGS

The accompanying drawings illustrate a number of exemplary embodiments and are a part of the specification. Together with the following description, these drawings demonstrate and explain various principles of the present disclosure.

FIG. 1 is a schematic diagram of components of an exemplary biosignal system in accordance with some embodiments of the technology described herein.

FIG. 2 is a simplified diagram of the biosignal system of FIG. 1 in accordance with some embodiments.

FIG. 3 is an illustration of exemplary regions of a composite bioelectrode in accordance with some embodiments.

FIG. 4 is an illustration of an exemplary layered configuration of the composite bioelectrodes of FIG. 1 in accordance with some embodiments.

FIG. 5 is a plot of an exemplary varying bioelectrode property in accordance with some embodiments.

FIG. 6 is a plot of an exemplary varying bioelectrode property in accordance with some embodiments.

FIG. 7 is an illustration of an exemplary configuration of the composite bioelectrodes of FIG. 1 in accordance with some embodiments.

FIG. 8 is a plot of an exemplary varying bioelectrode property in accordance with some embodiments.

FIG. 9 is a plot of an exemplary varying bioelectrode property in accordance with some embodiments.

FIG. 10 is an illustration of an exemplary configuration of the composite bioelectrodes of FIG. 1 in accordance with some embodiments.

FIG. 11 is a plot of an exemplary varying bioelectrode property in accordance with some embodiments.

FIG. 12 is an illustration of an exemplary configuration of the composite bioelectrodes of FIG. 1 in accordance with some embodiments.

FIG. 13 is an illustration of an exemplary configuration of the composite bioelectrodes of FIG. 1 in accordance with some embodiments.

FIG. 14 is an illustration of an exemplary configuration of the composite bioelectrodes of FIG. 1 in accordance with some embodiments.

FIG. 15 is an illustration of an exemplary configuration of the composite bioelectrodes of FIG. 1 in accordance with some embodiments.

FIG. 16 is an illustration of an exemplary monolithic bioelectrode array in accordance with some embodiments.

FIG. 17 is an illustration of an exemplary configuration of the monolithic bioelectrode array of FIG. 16 in accordance with some embodiments.

FIG. 18 is a side view of the exemplary configuration of FIG. 17 in accordance with some embodiments.

FIG. 19 is an illustration of another exemplary configuration of the monolithic bioelectrode array of FIG. 16 in accordance with some embodiments.

FIG. 20 is a flow diagram of an exemplary method for manufacturing composite bioelectrodes.

FIG. 21 is an illustration of an exemplary wristband system, according to at least one embodiment of the present disclosure.

FIG. 22 is a perspective view of another exemplary wristband system, according to at least one embodiment of the present disclosure.

FIG. 23 is an illustration of exemplary devices in accordance with some embodiments.

FIG. 24 is an illustration of exemplary devices in accordance with some embodiments.

FIG. 25 is an illustration of an exemplary artificial-reality headband that may be used in connection with embodiments of this disclosure.

FIG. 26 is an illustration of exemplary augmented-reality glasses that may be used in connection with embodiments of this disclosure.

FIG. 27 is a perspective view of an exemplary head-mounted display device in accordance with some embodiments.

FIG. 28 is an illustration of an exemplary virtual-reality headset that may be used in connection with embodiments of this disclosure.

FIG. 29 is an illustration of exemplary haptic devices that may be used in connection with embodiments of this disclosure.

FIG. 30 is an illustration of an exemplary virtual-reality environment according to embodiments of this disclosure.

FIG. 31 is an illustration of an exemplary augmented-reality environment according to embodiments of this disclosure.

Throughout the drawings, identical reference characters and descriptions indicate similar, but not necessarily identical, elements. While the exemplary embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the present disclosure covers all modifications, equivalents, and alternatives.

### DETAILED DESCRIPTION OF EXEMPLARY EMBODIMENTS

Obtaining consistent high-quality biosignals using conventional electrodes and conventional signal processing techniques is challenging, in part due to impedance mismatches at the interface between a user's skin and the electrodes. For applications that require near real-time analysis of biosignals, the acquisition of consistent high-quality signals is important to be able to iterate quickly on the recorded data. Poor electromechanical coupling of electrodes and the human body may greatly increase signal noise and may make captured biological signals unusable for analysis. Some conventional techniques for electromechanically coupling electrodes and the human body include the use of wet electrodes or the use of dry electrodes in combination with skin preparations (e.g., shaving, sanding, or hydrating with cream). Other conventional techniques for electromechanically coupling electrodes and the human body include using adhesives or pressing the electrodes against users' skin, which may be inconvenient and/or cause discomfort for the users. In spite of these techniques, conventional electrodes tend to have considerable variability in contact impedance caused by variations at the electrode-skin interface.

The present disclosure details various exemplary designs and configurations of composite bioelectrodes for biological-signal measurement; in particular, soft and flexible designs and configurations intended for use in wristbands. The disclosed composite bioelectrodes may be engineered with varying mechanical and/or electrical properties to improve electromechanical coupling between (1) the composite bioelectrodes and users' skin and/or (2) the composite bioelectrodes and other electrical or mechanical components. Various properties of the disclosed composite bioelectrodes may be engineered to better control for (1) electrode-skin electrical impedance changes caused by motion between the electrodes and the users' skin, (2) changes in contact area between composite bioelectrodes and the users' skin, and/or (3) other unintended electromechanical changes that effect electrode-skin electrical impedances.

In some embodiments, the disclosed composite bioelectrodes may be optimized for effective contact area, improved biological-signal quality, and/or comfort by tuning their electrical properties, mechanical properties, and/or geometries to match the electrical properties, mechanical properties, and/or geometries of specific body locations to which the composite bioelectrodes will interface. Additionally or alternatively, the disclosed composite bioelectrodes may be optimized for improved biological-signal quality and/or acquisition density by tuning their electrical properties, mechanical properties, and/or geometries to match the electrical properties, mechanical properties, and/or geometries of the transducer and device components to which the composite bioelectrodes will interface and/or connect. In some embodiments, the disclosed composite bioelectrodes may be monolithically created by layering and/or patterning multiple regions with varying properties (e.g., using anisotropically conductive polymers). The disclosed processes for creating composite bioelectrodes may extend to large one-dimensional and/or two-dimensional arrays of composite bioelectrodes (e.g., by patterning multiple areas of conductive and non-conductive materials within a single monolithic structure).

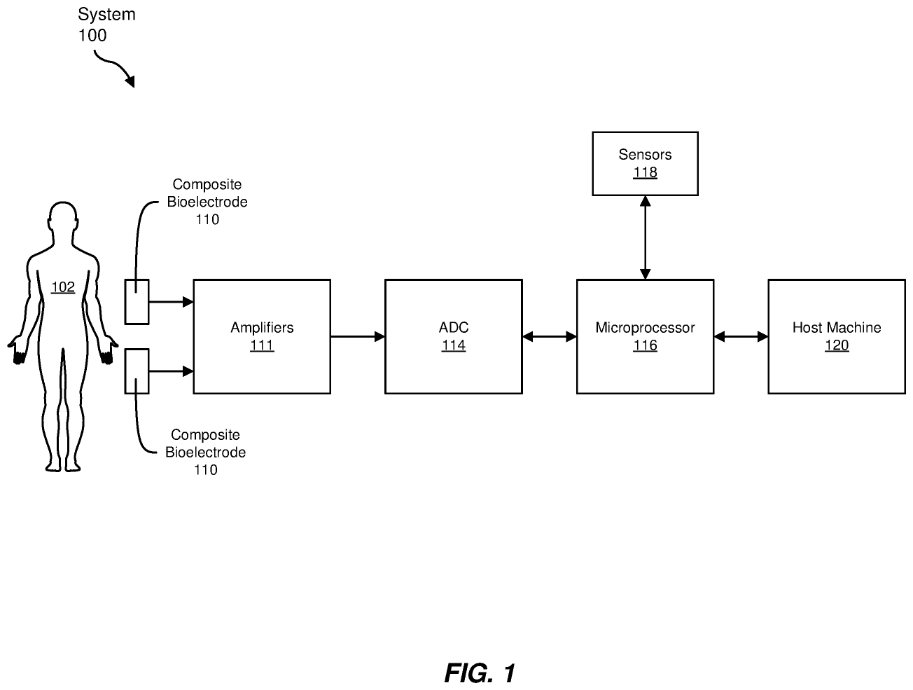

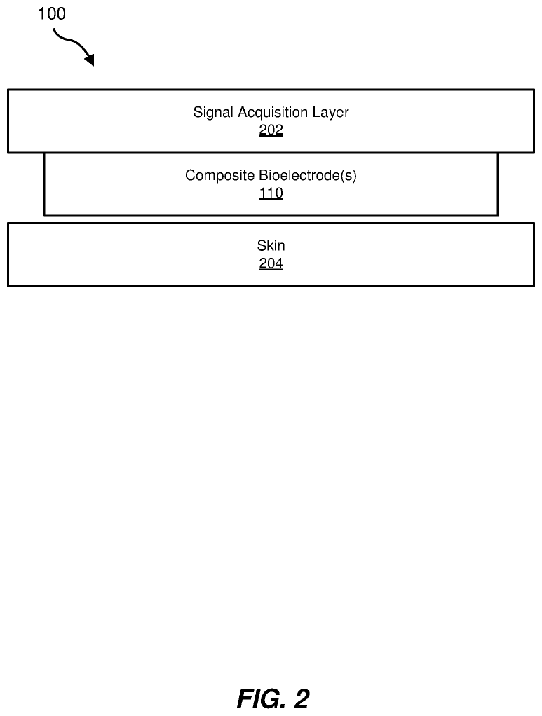

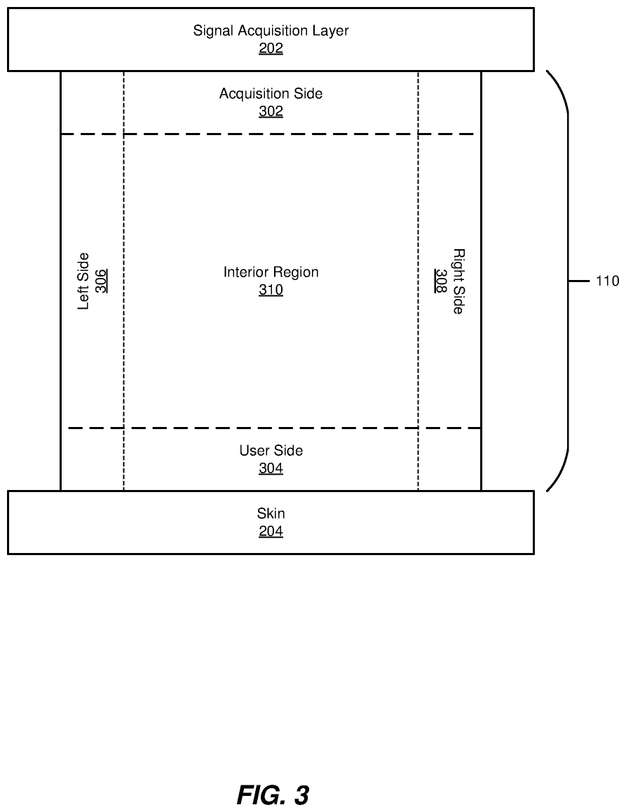

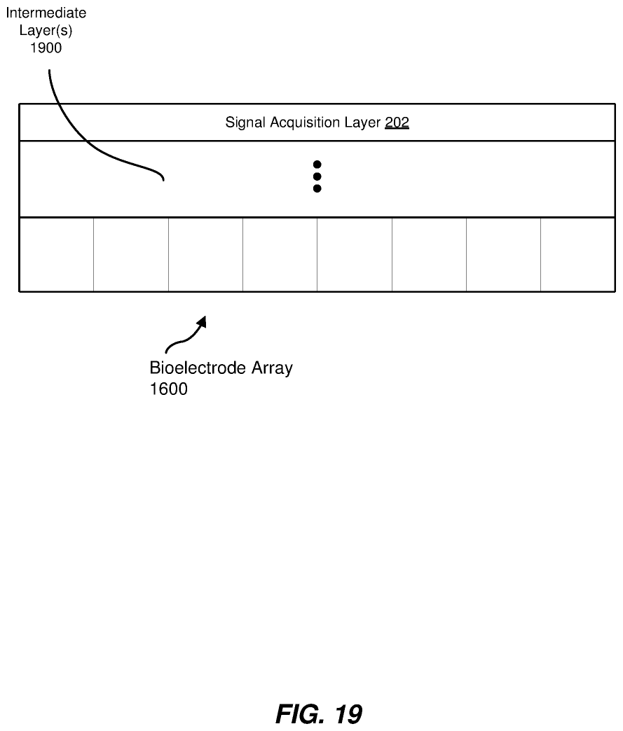

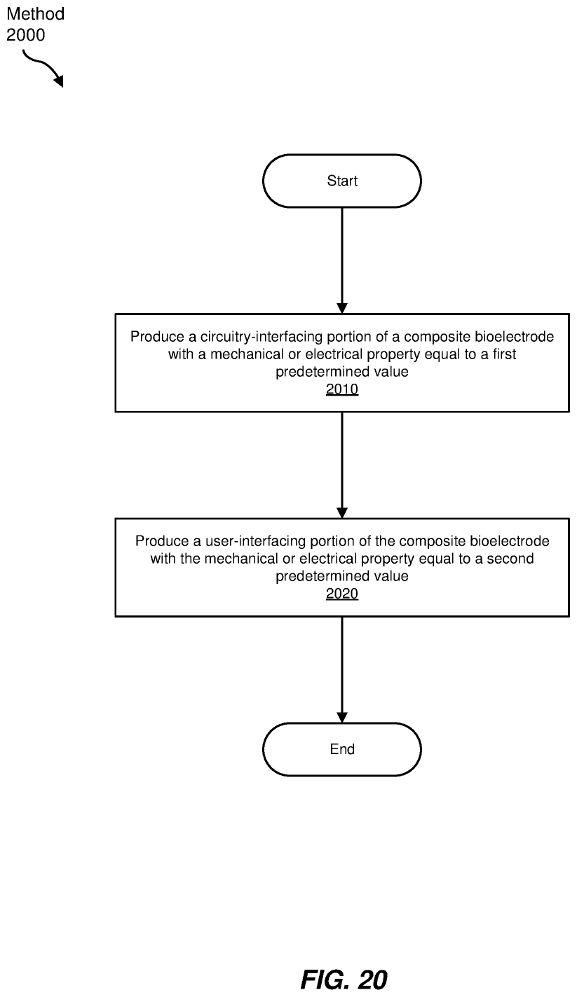

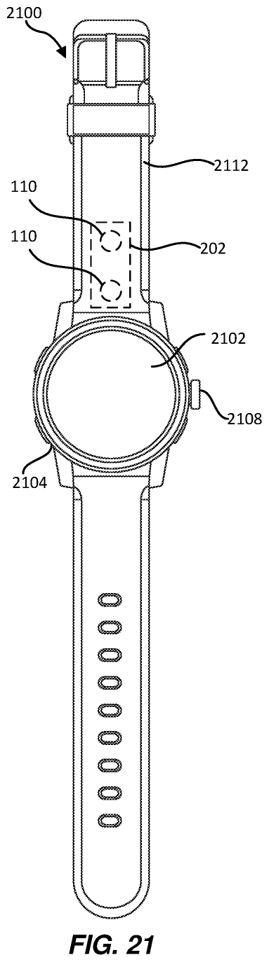

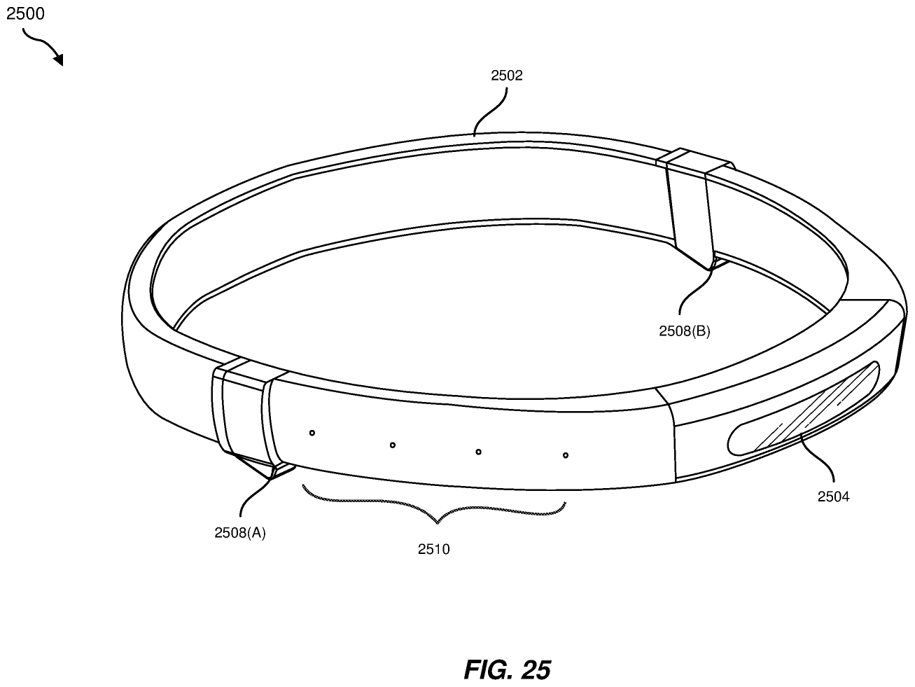

The following will provide, with reference to FIGS. 1 and 2, detailed descriptions of an exemplary biosignal system. The descriptions corresponding to FIGS. 3-19 provide detailed descriptions of exemplary composite bioelectrodes. The discussion corresponding to FIG. 20 will provide examples of methods for manufacturing, configuring, and/or optimizing the composite bioelectrodes presented herein. The descriptions corresponding to FIGS. 21-24 will provide examples of systems implementing embodiments of the composite bioelectrodes presented herein. Finally, with reference to FIGS. 25-31, the following will provide detailed descriptions of various artificial-reality systems and components that may implement embodiments of the present disclosure.

FIG. 1 schematically illustrates components of a biosignal system 100 in accordance with some embodiments. System 100 includes a pair of composite bioelectrodes 110 configured to register or measure a biosignal (e.g., an Electrooculography (EOG) signal, an Electromyography (EMG) signal, an Electroencephalography (EEG) signal, an Electrocardiography (ECG) signal, etc.) generated by the body of a user 102 (e.g., for electrophysiological monitoring or stimulation). In some embodiments, composite bioelectrodes 110 may be arranged as a portion of a wearable device configured to be worn on or around part of a user's body. For example, in one nonlimiting example, a plurality of composite bioelectrodes including composite bioelectrodes 110 may be arranged circumferentially around an adjustable and/or elastic band such as a wristband or armband configured to be worn around a user's wrist or arm. Additionally or alternatively, at least some of composite bioelectrodes 110 may be arranged on a wearable patch configured to be affixed to or placed in contact with a portion of the body of user 102. It should be appreciated that any suitable number of composite bioelectrodes may be used, and the number and arrangement of composite bioelectrodes may depend on the particular application for which a device is used.

Surface potentials measured or recorded by composite bioelectrodes 110 may be small, and amplification of the biosignals recorded by composite bioelectrodes 110 may be desired. As shown in FIG. 1, composite bioelectrodes 110 may be coupled to amplification circuitry 111 configured to amplify the biosignals conducted by composite bioelectrodes 110. The output of amplification circuitry 111 may be provided to analog-to-digital converter (ADC) circuitry 114, which may convert the amplified biosignals to digital signals for further processing by a microprocessor 116. Microprocessor 116 may be implemented by one or more hardware processors. In some embodiments, composite bioelectrodes 110, amplification circuitry 111, ADC circuitry 114, and/or microprocessor 116 may represent some or all of a biosignal sensor. The processed signals output from microprocessor 116 may be interpreted by a host machine 120, examples of which include, but are not limited to, a desktop computer, a laptop computer, a smartwatch, a smartphone, a head-mounted display device, or any other computing device. In some implementations, host machine 120 may be configured to output one or more control signals for controlling a physical or virtual device based, at least in part, on an analysis of the signals output from microprocessor 116. As shown, biosignal system 100 may include additional sensors 118, which may be configured to record types of information about a state of a user other than biosignal information. For example, sensors 118 may include, temperature sensors configured to measure skin/electrode temperature, inertial measurement unit (IMU) sensors configured to measure movement information such as rotation and acceleration, humidity sensors, and other bio-chemical sensors configured to provide information about the user and/or the user's environment.

FIG. 2 is a simplified illustration of biosignal system 100. As shown, biosignal system 100 includes a signal acquisition layer 202 to which composite bioelectrode(s) 110 may be mechanically and/or electrically coupled. In some embodiments, signal acquisition layer 202 may represent some or all of amplification circuitry 111, some or all of ADC circuitry 114, some or all of microprocessor 116, some or all of sensors 118, some or all of host machine 120, and/or any other associated component (e.g., flexible electronics, connectors, wiring, housings, mounts, supports, substrates, etc.). In some examples, signal acquisition layer 202 may include a Printed Circuit Board (PCB), a Printed Circuit Board Assembly (PCBA), and/or a Flexible Printed Circuit (FPC). As shown, composite bioelectrode(s) 110 may be mechanically and electrically coupled to all or a portion of signal acquisition layer 202 and/or may be configured to interface with an area of skin 204 of user 102.

FIG. 3 illustrates exemplary regions of composite bioelectrode(s) 110. As shown, composite bioelectrode(s) 110 may have an acquisition side 302 that is mechanically and/or electrically coupled to signal acquisition layer 202, a user side 304 that is configured to mechanically and electrically interface with an area of skin 204, a left side 306, a right side 308, and an interior region 310. As will be explained in greater detail below, composite bioelectrode(s) 110 may have mechanical and/or electrical properties that vary (e.g., differ in size, amount, degree, or nature) from one region of composite bioelectrode(s) 110 to another and/or may vary across regions of composite bioelectrode(s) 110. In some embodiments, a mechanical and/or electrical property of composite bioelectrode(s) 110 may vary from acquisition side 302 to user side 304. In other embodiments, the mechanical and/or electrical property may vary from left side 306 to right side 308. In other embodiments, the mechanical and/or electrical property may vary from acquisition side 302 to user side 304 while also varying from left side 306 to right side 308.

In some embodiments, composite bioelectrode(s) 110 may have one or more mechanical and/or electrical properties that continuously vary from one region to another. In other embodiments, composite bioelectrode(s) 110 may have one or more mechanical and/or electrical properties that discontinuously vary from one region or side to another. In some embodiments, a property of composite bioelectrode(s) 110 may monotonically increase from one region to another. Additionally or alternatively, a property of composite bioelectrode(s) 110 may monotonically decrease from one region to another. In at least one embodiment, a property of composite bioelectrode(s) 110 may vary from one region to another in a way that is not monotonic.

Examples of mechanical properties that may vary from one region of composite bioelectrode(s) 110 to another include, without limitation, softness, hardness, durometry, flexibility, pliability, plasticity, elasticity, stiffness, rigidity, springiness, compressibility, resilience, viscosity, dampening, anisotropy, thermal conductivity, thermal expansion, shape, geometry, concavity, convexity, surface area, cross-sectional area, volume, dimension, texture, tackiness, stickiness, density, tactile feedback, material, material distribution, and material concentration. Examples of electrical properties that may vary from one region of composite bioelectrode(s) 110 to another include, without limitation, impedance, capacitance, resistivity, conductivity, anisotropy of conduction, and electrostriction.

In some embodiments, a mechanical and/or electrical property of acquisition side 302 may have a value equal to or matching the value of the mechanical and/or electrical property of signal acquisition layer 202. For example, a stiffness or softness of acquisition side 302 may be equal to the stiffness or softness of signal acquisition layer 202. In some embodiments, a mechanical and/or electrical property of user side 304 may have a value equal to or matching a value of the same mechanical and/or electrical property of skin 204. For example, a stiffness or softness of user side 304 may be equal to the stiffness or softness of skin 204.

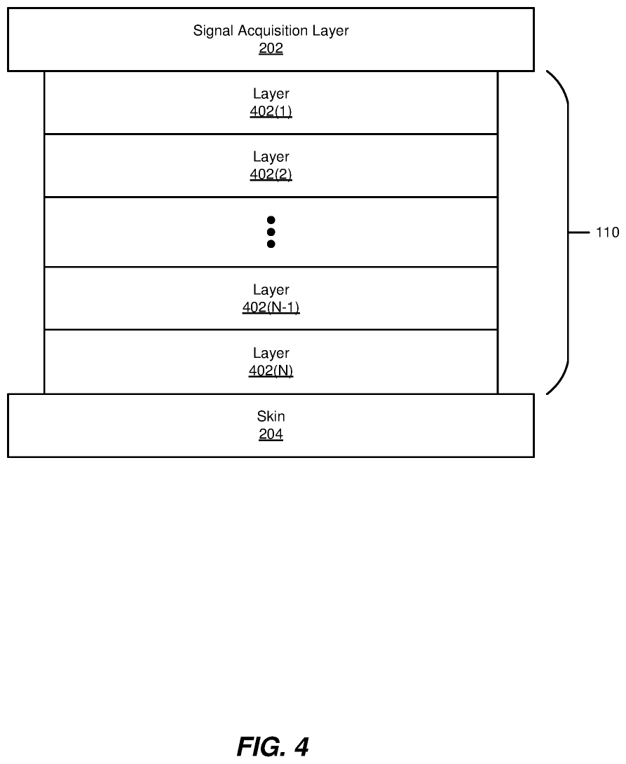

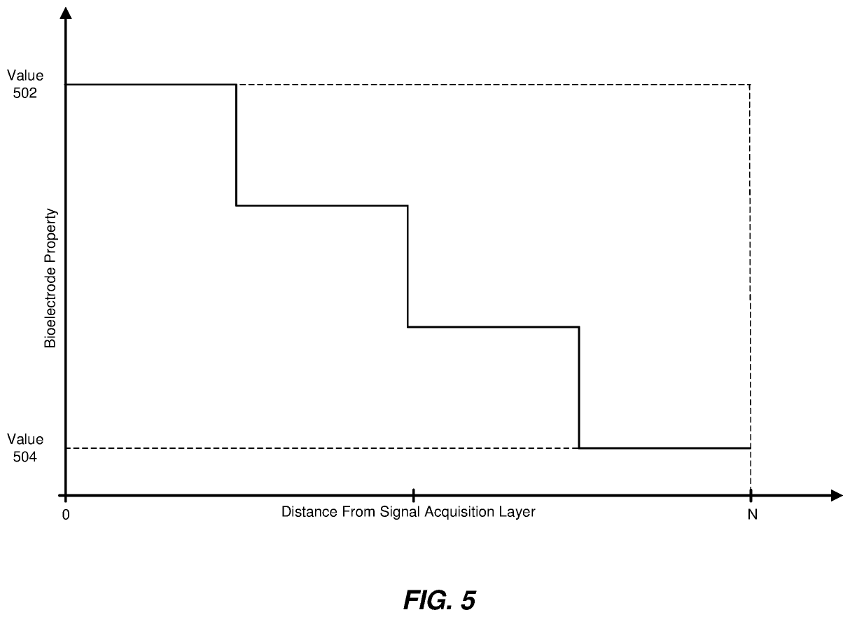

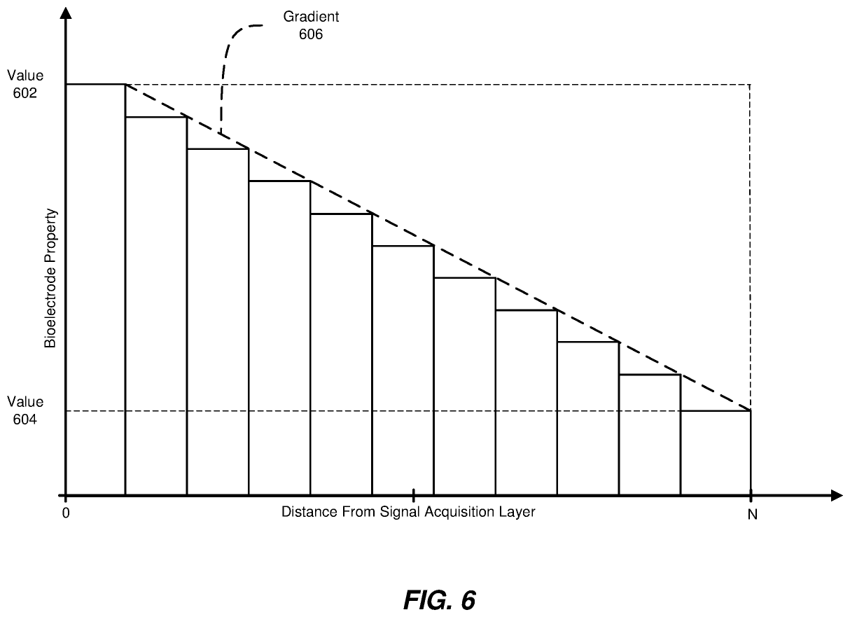

FIG. 4 is an illustration of an exemplary configuration for bioelectrode 110 having distinguishable horizontal layers 402(1)-(N). In some embodiments, one or more of layers 402(1)-(N) may have a mechanical and/or electrical property equal to a single value. In at least one embodiment, one or more of layers 402(1)-(N) may have a mechanical and/or electrical property whose value varies across the layer. In some embodiments, one or more mechanical and/or electrical properties of layers 402(1)-(N) may vary discontinuously from one layer to another. For example, as shown in FIG. 5, a property of layers 402(1)-(N) may vary discontinuously from value 502 to value 504. Additionally or alternatively, one or more mechanical and/or electrical properties of layers 402(1)-(N) may vary continuously from one layer to another. In some embodiments, one or more mechanical and/or electrical properties of layers 402(1)-(N) may vary discontinuously from one layer to another in sufficiently small increments in order to approximate a continuously varying or gradient property. For example, as shown in FIG. 6, a property of layers 402(1)-(N) may vary discontinuously from value 602 to value 604 to approximate gradient 606.

In one embodiment, layer 402(1) may be composed of a stiff conductive material, layer 402(N) may be composed of a soft conductive material, and layers 402(2)-402(N−1) may have monotonically decreasing stiffnesses ranging between the stiffness of layer 402(1) and the softness of layer 402(N). In some embodiments, the stiffness of layer 402 may be substantially equal to a stiffness of signal acquisition layer 202, and/or the softness of layer 402(N) may be equal to a softness of skin 204.

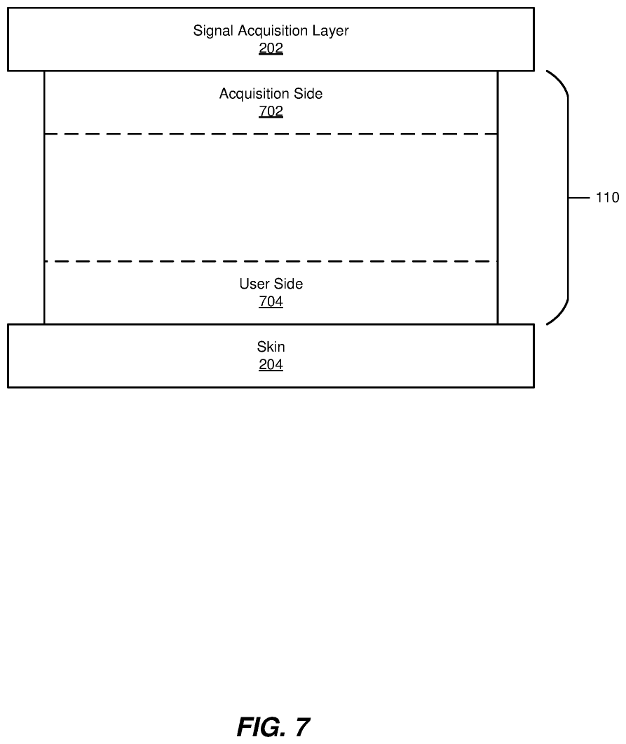

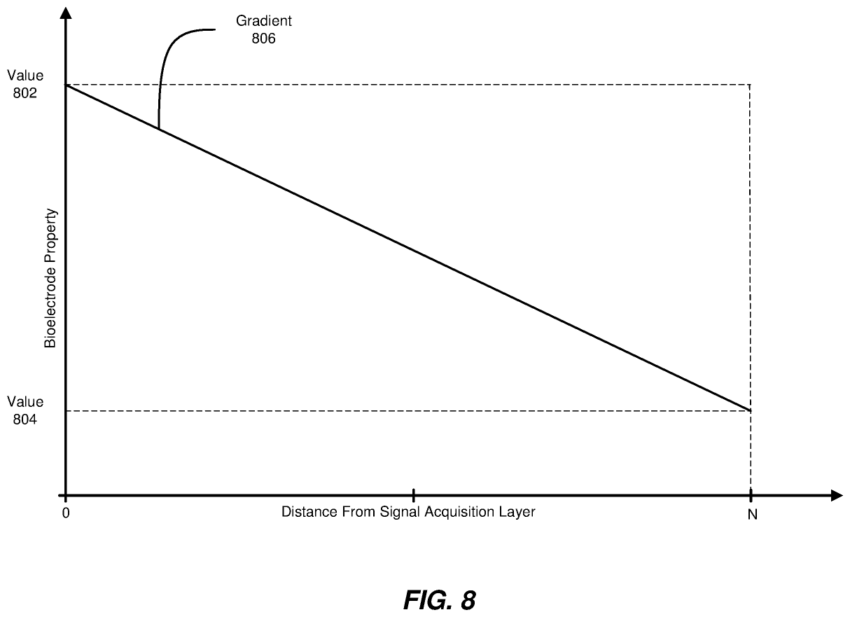

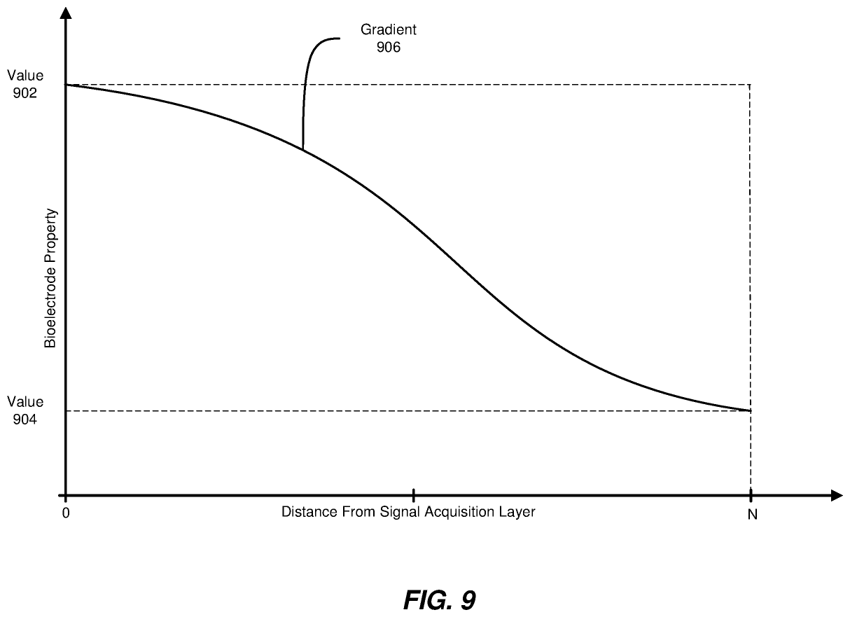

FIG. 7 is an illustration of an exemplary configuration for composite bioelectrode 110 having no distinguishable layers. In some embodiments, a mechanical and/or electrical property of composite bioelectrode 110 may vary continuously across bioelectrode 110 from one value at acquisition side 702 to another value at user side 704. In some embodiments, the property may monotonically increase from one value at acquisition side 702 to another value at user side 704. Alternatively, the property of composite bioelectrode 110 may monotonically decrease from one value at acquisition side 702 to another value at user side 704. In at least one embodiment, the property of composite bioelectrode 110 may vary from one value at acquisition side 702 to another value at user side 704 in a way that is not monotonic. In some embodiments, a property may monotonically increase or decrease at a constant rate. For example, as shown in FIG. 8, the property may vary at a constant rate from a value 802 at acquisition side 702 to value 804 at user side 704 along gradient 806. In other embodiments, the property may monotonically increase or decrease at a variable rate. For example, as shown in FIG. 9, the property may vary at a variable rate from a value 902 at acquisition side 702 to a value 904 at user side 704 along gradient 906. In some embodiments, a property of acquisition side 702 may have a value that matches a value of the property of signal acquisition layer 202, and/or the property of user side 704 may have a value that matches a value of the property of skin 204. For example, acquisition side 702 may have a stiffness substantially equal to a stiffness of signal acquisition layer 202, and/or user side 704 may have a softness substantially equal to a softness of skin 204.

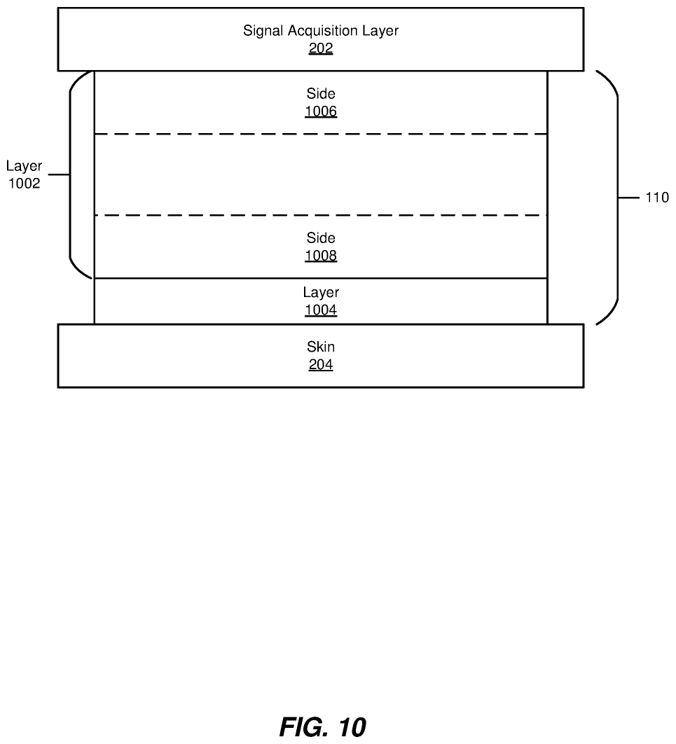

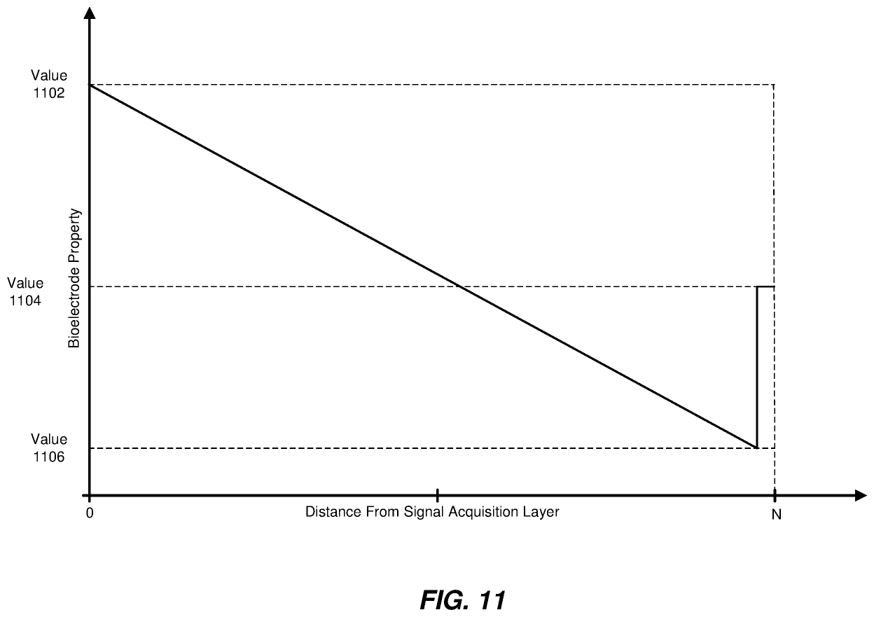

FIG. 10 is an illustration of an exemplary configuration for bioelectrode 110 having two distinguishable layers (i.e., a layer 1002 and a layer 1004). In this embodiment, layers 1002 may have a mechanical and/or electrical property whose value varies across layer 1002, while layer 1004 may have a single value for the same mechanical and/or electrical property. In one example, as shown in FIG. 11, a property of layer 1002 may vary continuously from value 1102 to value 1106, and the property of layer 1004 may have a single value equal to value 1104. In some embodiments, value 1102 may equal a stiffness of signal acquisition layer 202, and/or value 1104 may equal a softness of skin 204.

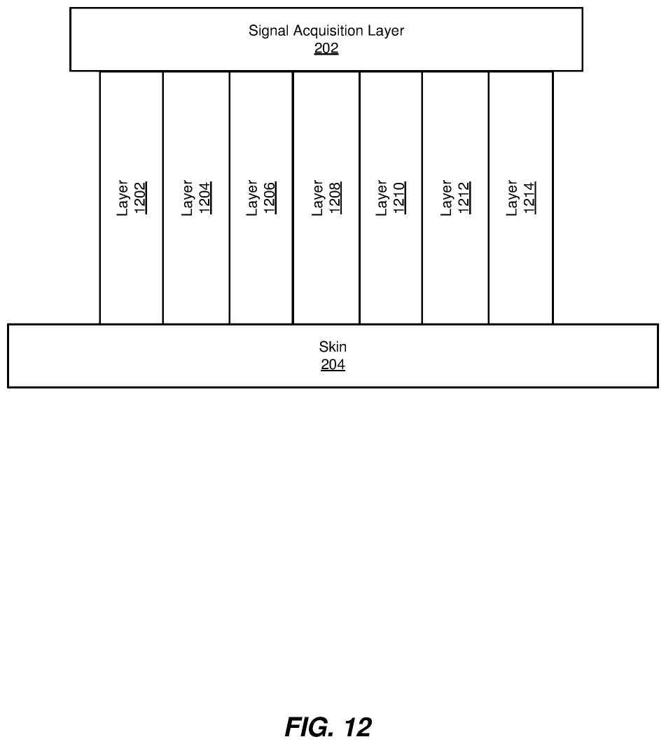

FIG. 12 is an illustration of an exemplary configuration for bioelectrode 110 having multiple vertical layers (i.e., layers 1202-1214). Mechanical and/or electrical properties of layers 1202-1214 may vary in a manner similar to the variations of the mechanical and/or electrical properties of the horizontal layers described above. In one embodiment, a mechanical and/or electrical property of vertical layers 1202-1214 may vary discontinuously in an alternating manner. For example, vertical layers 1202, 1206, 1210, and 1214 may be composed of a stiff conductive material, and vertical layers 1204, 1208, and 1212 may be composed of a soft nonconductive material.

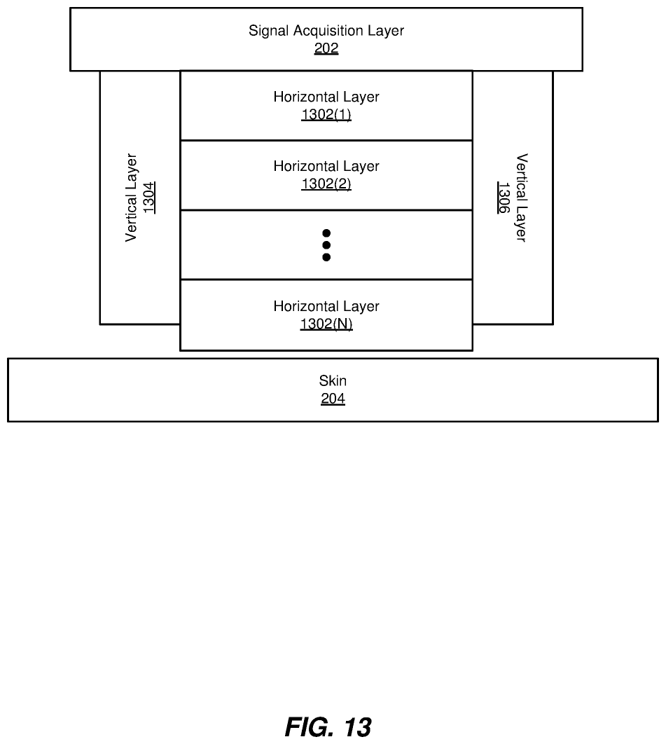

FIG. 13 is an illustration of an exemplary configuration for bioelectrode 110 having multiple horizontal layers (i.e., layers 1302(1)-(N)) and multiple vertical layers (i.e., layers 1304 and 1306). Mechanical and/or electrical properties of layers 1302(1)-(N) and layers 1304 and 1306 may vary in a manner similar to the variations described above. In one embodiment, layers 1302(1)-(N) may be composed of a conductive material, and layers 1304 and 1306 may be composed of a nonconductive material. Additionally or alternatively, layers 1302(1)-(N) may vary in stiffness (e.g., layer 1302(1) may have a stiffness equal to a stiffness of signal acquisition layer 202, and layer 1302(N) may have a softness equal to a softness of skin 204), and layers 1304 and 1306 may be equal in stiffness.

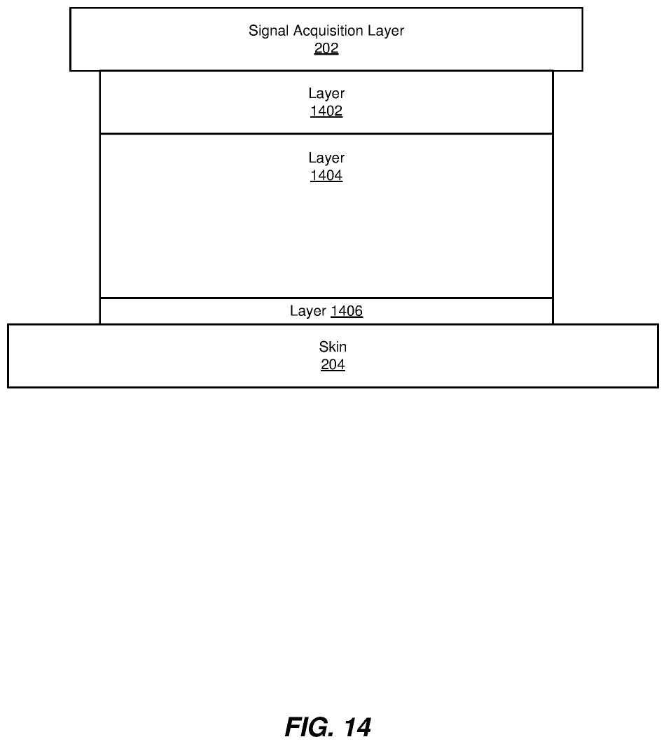

FIG. 14 is an illustration of an exemplary configuration for bioelectrode 110 having multiple horizontal layers 1402-1406 with different thicknesses. Other mechanical and/or electrical properties of layers 1402-1406 may vary in a manner similar to the variations described above. In one embodiment, layer 1404 may be soft relative to layers 1402 and 1406. Additionally or alternatively, layer 1402 may have a stiffness equal to a stiffness of signal acquisition layer 202, and/or layer 1406 may have a softness equal to a softness of skin 204.

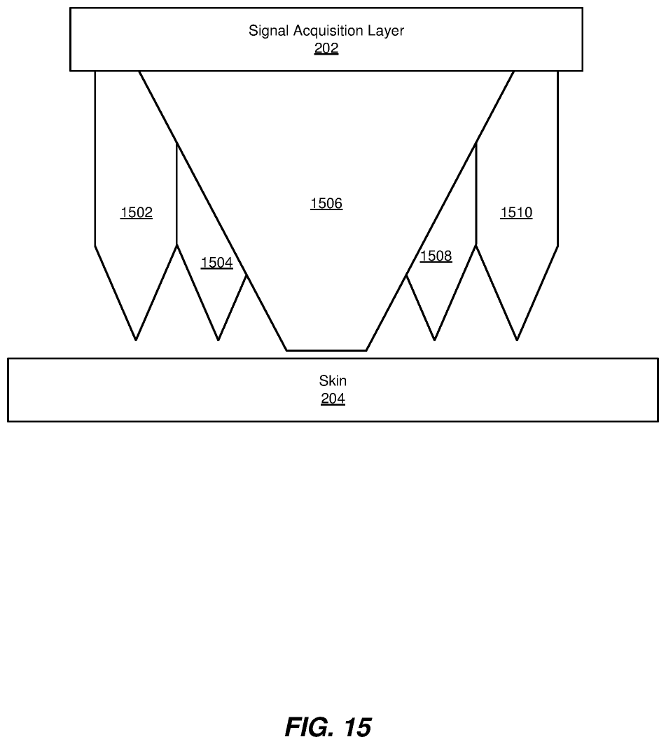

FIG. 15 is an illustration of an exemplary configuration for bioelectrode 110 having multiple regions 1502-1510 with differing shapes, volumes, surfaces, etc. Other mechanical and/or electrical properties of regions 1502-1510 may vary in a manner similar to the variations described above. In one embodiment, regions 1502-1510 may be composed of a conductive material, and region 1506 may be stiff relative to regions 1502, 1504, 1508, and 1510.

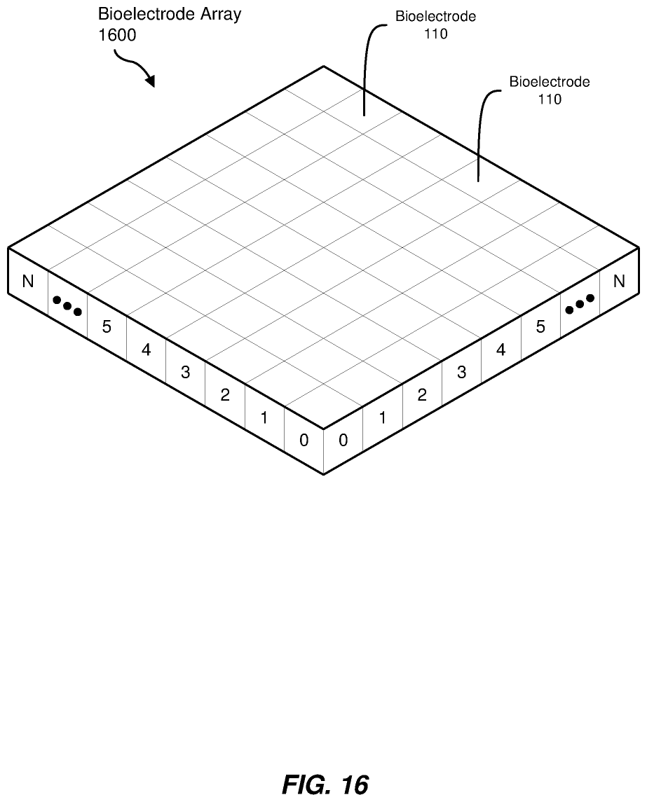

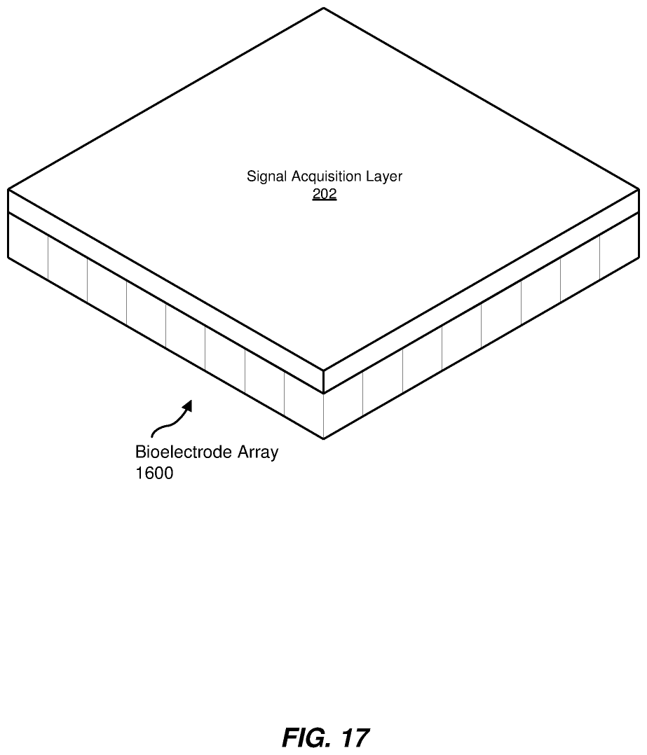

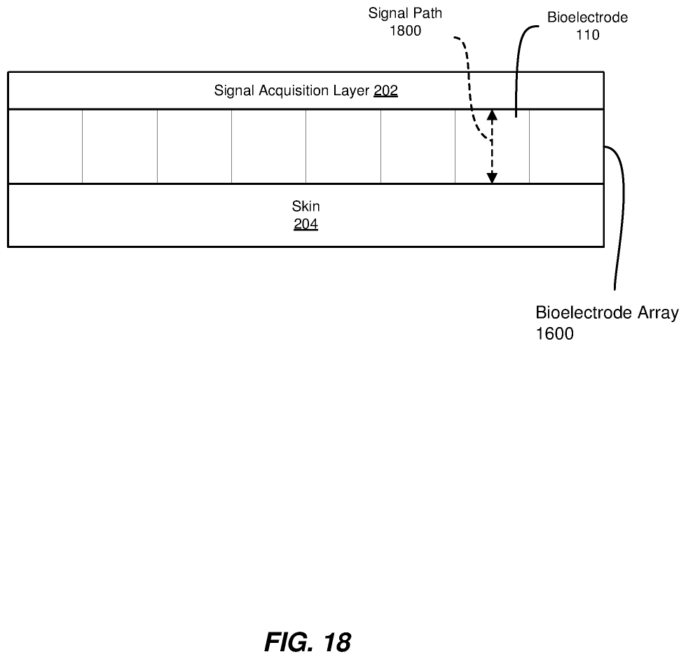

Composite bioelectrodes 110 may be produced individually or together as a single monolithic or bulk structure. FIGS. 16-19 illustrate several monolithic exemplary configurations for bioelectrodes 110. As shown in FIG. 16, composite bioelectrodes 110 may form a bioelectrode array 1600 having bioelectrodes 110 arranged in an N×N grid. As shown in FIGS. 17 and 18, bioelectrode array 1600 may be mechanically coupled to signal acquisition layer 202. In the configurations illustrated in these figures, the mechanical and/or electrical properties of each of bioelectrodes 110 may be varied in a manner similar to the variations described above. In some embodiments, each of bioelectrodes 110 in bioelectrode array 1600 may be separated by a nonconductive material or may be composed of an anisotropically conductive material such that each of each of bioelectrodes 110 provides a signal path or channel (e.g., signal path 1800) between electrical connections in signal acquisition layer 202 and skin 204. In some embodiments, bioelectrode array 1600 may form a portion of a wristband or other wearable device and/or may include tens of channels to conduct tens of biosignals or hundreds of channels to conduct hundreds of biosignals. In some embodiments, bioelectrode array 1600 may be used to redundantly measure biosignals and/or detect a direction of travel of a particular biosignal. In some embodiments, bioelectrode array 1600 may be mechanically coupled to signal acquisition layer 202 via one or more intermediate layers 1900. In at least one embodiment, each of intermediate layers 1900 may be a bioelectrode array.

FIG. 20 is a flow diagram of an exemplary manufacturing method 2000 for producing composite bioelectrodes according to any of the embodiments disclosed herein. The steps shown in FIG. 20 may be performed by an individual and/or by any suitable manual and/or automated apparatus. As illustrated in FIG. 20, at step 2010, a circuitry-interfacing portion of a composite bioelectrode may be produced with a mechanical or electrical property equal to a first predetermined value. Then at step 2020, a user-interfacing portion of the composite bioelectrode may be produced with the mechanical or electrical property equal to a second predetermined value. In some embodiments, additional portions of the composite bioelectrode may be produced with the mechanical or electrical property being equal to other predetermined values. Using FIG. 4 as an example, layer 402(1) may be produced with a mechanical or electrical property equal to a first predetermined value, and layer 402(n) may be produced with a mechanical or electrical property equal to a second predetermined value. Using FIG. 7 as another example, acquisition side 702 may be produced with a mechanical or electrical property equal to a first predetermined value, and user side 704 may be produced with a mechanical or electrical property equal to a second predetermined value.

A composite bioelectrode may be produce with varying mechanical and/or electrical properties in a variety of ways. In some embodiments, mechanical and/or electrical properties may be varied by using multiple materials with differing mechanical and/or electrical properties. Additionally or alternatively, mechanical and/or electrical properties may be varied using multiple combinations of materials, each combination having a different mechanical and/or electrical property. In some embodiments, mechanical and/or electrical properties may be varied by varying how each portion of a composite bioelectrode is processed (e.g., by varying cure times, mixing times, temperatures, etc.).

Examples of materials that may be used to produce the composite bioelectrodes described herein include, without limitation, elastomers, polymers, silicones (e.g., platinum-cured silicones), fluoroelastomers, fluorosilicones, fluoro-terpolymers, thermo plastic elastomers, thermoplastic polyurethanes, variably conductive materials or combinations of materials, materials or combinations of materials with variable durometry, anisotropic materials, and/or any combination thereof. The composite bioelectrodes described herein may be formed from any suitable electrically conductive material or combination of electrically conductive materials. In some examples, all or a portion of a composite bioelectrode may be nonconductive.

The composite bioelectrodes described herein may be produced using various manufacturing processes. For example, the composite bioelectrodes described herein may be produced using injection molding, multi-shot Injection molding, compression molding, overmolding, screen printing, stencil printing, injet printing, multi-layer lamination, laser ablation, patterning processes (e.g., photolithography), etching, three-dimensional printing, multi-head dispensing, doping, and/or any combination thereof.

In some embodiments, certain mechanical and/or electrical properties of a composite electrode may remain constant across the composite electrode, while other mechanical and/or electrical properties of the composite electrode may be varied across the composite electrode. For example, a composite bioelectrode may be formed from one or more materials such that a conductivity of the composite bioelectrode remains constant across the composite electrode while a stiffness of the composite electrode may be varied across the composite electrode.

The mechanical and/or electrical properties of the composite bioelectrodes described herein may be optimized for a variety of purposes. For example, the mechanical and/or electrical properties of all or a portion of a composite bioelectrode may be optimized to minimize contact impedances for a particular frequency, a particular range of frequencies (e.g., a range of tens of hertz to thousands of hertz), and/or a particular current density of interest. In some embodiments, the mechanical and/or electrical properties of a portion of a composite bioelectrode that contacts or is in close proximity to a user's skin may be selected or varied to minimize electrode-skin contact impedance, to maximize user comfort, to maximize contact area, and/or to maximize robustness. Additionally or alternatively, the mechanical and/or electrical properties of a portion of a composite bioelectrode that contacts signal acquiring circuitry may be selected or varied to minimize electrode-circuitry contact impedance and/or maximize connection durability. In at least one embodiment, the mechanical and/or electrical properties of portions of a composite bioelectrode may be selected or varied to minimize the effects of compression on the conductivity of the composite bioelectrode.

Embodiments of the present disclosure may include or be implemented in conjunction with various types of wearable devices. FIG. 21 illustrates an example system 2100 that includes a watch body 2104 coupled to a wristband 2112. Watch body 2104 and wristband 2112 may have any size and/or shape that is configured to allow a user to wear system 2100 on a body part (e.g., a wrist). System 2100 may perform various functions associated with the user. The functions may be executed independently in watch body 2104, independently in wristband 2112, and/or in communication between watch body 2104 and wristband 2112. Functions executed by system 2100 may include, without limitation, display of visual content to the user (e.g., visual content displayed on display screen 2102), sensing user input (e.g., sensing a touch on button 2108, sensing biometric data or neuromuscular signals with composite bioelectrodes 110, messaging (e.g., text, speech, video, etc.), image capture, wireless communications (e.g., cellular, near field, WiFi, personal area network, etc.), location determination, financial transactions, providing haptic feedback, etc. Functions may be executed on system 2100 in conjunction with an artificial-reality system.

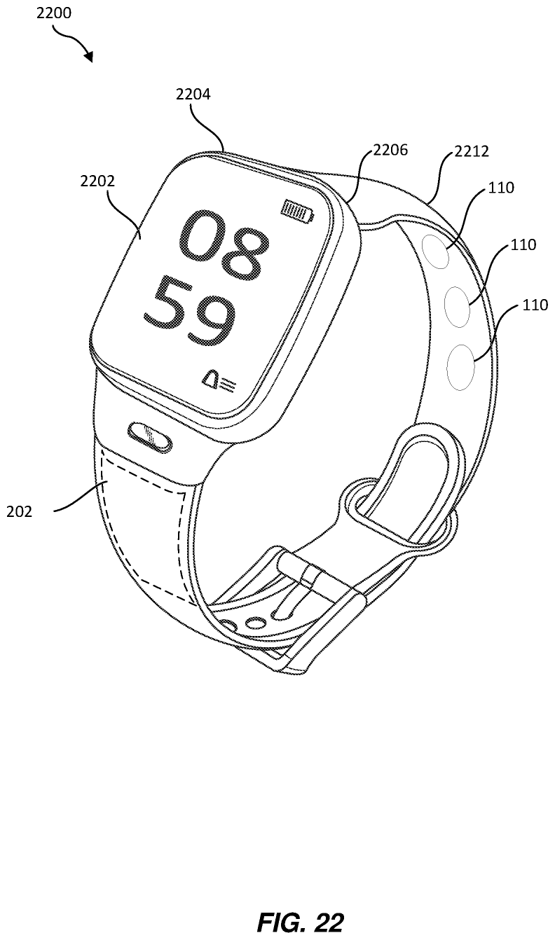

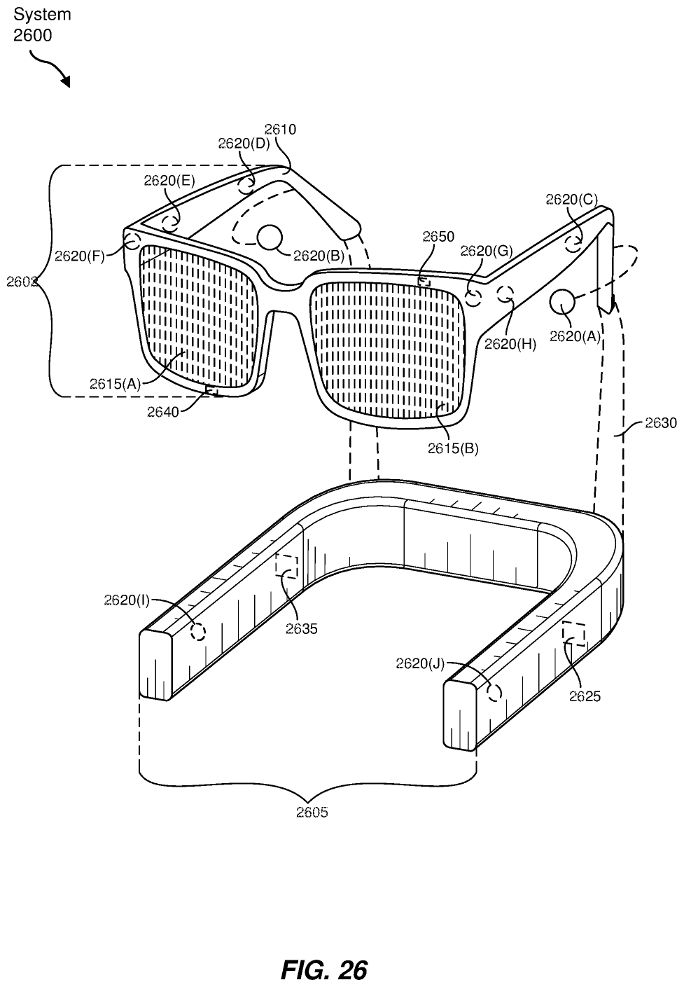

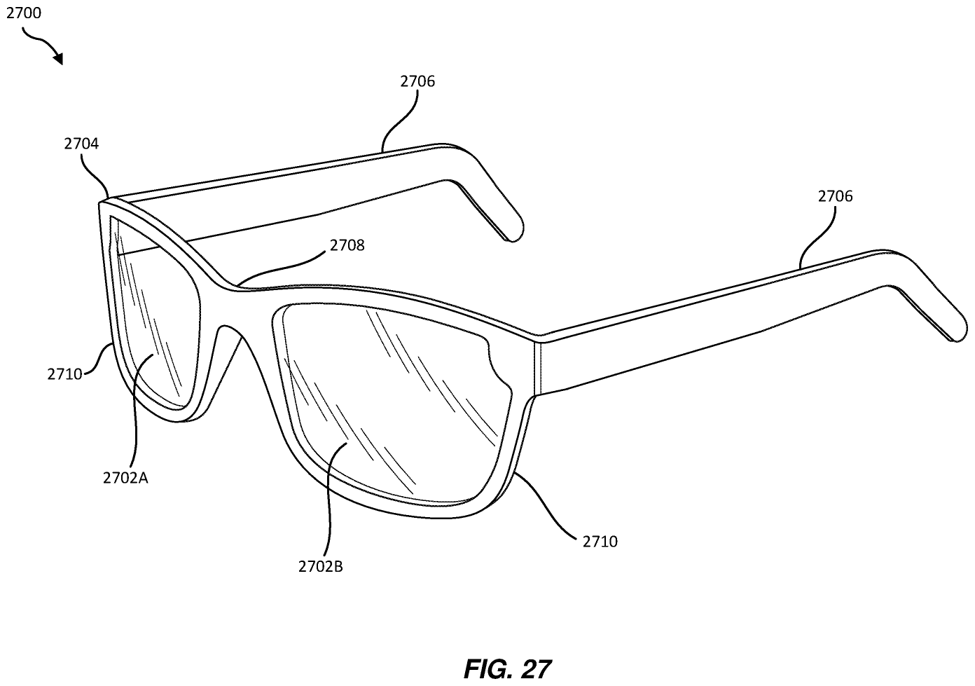

Wristband 2112 may be donned (e.g., worn) on a body part (e.g., a wrist) of a user and may operate independently from watch body 2104. For example, wristband 2112 may be configured to be worn by a user and an inner surface of wristband 2112 may be in contact with the user's skin. When worn by a user, composite bioelectrodes 110 may be in contact with the user's skin. As described in detail below with reference to FIG. 22, an electromyography sensor integrated into wristband 112 may sense a user's muscle intention. The sensed muscle intention may be transmitted to an artificial-reality system (e.g., the augmented-reality system 2600 in FIG. 26 or the virtual-reality system 2700 in FIG. 27) to perform an action in an associated artificial-reality environment, such as to control a physical and/or virtual object displayed to the user.

FIG. 22 illustrates a perspective view of an example wristband system 2200 that includes a watch body 2204 coupled to a wristband 2212. Wristband system 2200 may be structured and/or function similarly to wristband system 2100 of FIG. 21. Watch body 2204 and wristband 2212 may have a substantially rectangular or circular shape and may be configured to allow a user to wear wristband system 2200 on a body part (e.g., a wrist). Wristband system 2200 may perform various functions associated with the user as described above with reference to FIG. 21. Example functions executed by wristband system 2200 may include, without limitation, display of visual content to the user (e.g., visual content displayed on display screen 2202), sensing biometric data via composite bioelectrodes 110, sensing neuromuscular signals via composite bioelectrodes 110, messaging (e.g., text, speech, video, etc.), image capture, wireless communications (e.g., cellular, near field, WiFi, personal area network, etc.), location determination, financial transactions, providing haptic feedback, etc. These functions may be executed independently in watch body 2204, independently in wristband 2212, and/or in communication between watch body 2204 and wristband 2212. Functions may be executed on wristband system 2200 in conjunction with an artificial-reality system such as the artificial-reality systems described in FIGS. 25-31.

Wristband 2212 may be configured to be worn by a user such that an inner surface of wristband 2212 may be in contact with the user's skin. When worn by a user, composite bioelectrodes 110 may be in contact with the user's skin. Wristband 2212 may transmit the data acquired by composite bioelectrodes 110 to watch body 2204 using a wired communication method and/or a wireless communication method. Wristband 2212 may be configured to operate (e.g., to collect data using composite bioelectrodes 110) independent of whether watch body 2204 is coupled to or decoupled from wristband 2212.

In some examples, wristband 2212 may include signal acquisition circuitry 202. In some examples signal acquisition circuitry 202 may sense a user's muscle intention. The sensed muscle intention may be transmitted to an artificial-reality (AR) system to perform an action in an associated artificial-reality environment, such as to control the motion of a virtual device displayed to the user. Further, the artificial-reality system may provide haptic feedback to the user in coordination with the artificial-reality application via a haptic device. Signals from signal acquisition circuitry 202 may be used to provide a user with an enhanced interaction with a physical object and/or a virtual object in an AR environment generated by an AR system. Signals from signal acquisition circuitry 202 may be obtained (e.g., sensed and recorded) through one or more of composite bioelectrodes 110. In some examples, wristband 2212 may include a plurality of composite bioelectrodes 110 arranged circumferentially on an inside surface of wristband 2212 such that the plurality of composite bioelectrodes 110 contact the skin of the user. Signal acquisition circuitry 202 may sense and record neuromuscular signals from the user as the user performs muscular activations (e.g., movements, gestures, etc.). The muscular activations performed by the user may include static gestures, such as placing the user's hand palm down on a table; dynamic gestures, such as grasping a physical or virtual object; and covert gestures that are imperceptible to another person, such as slightly tensing a joint by co-contracting opposing muscles or using sub-muscular activations. The muscular activations performed by the user may include symbolic gestures (e.g., gestures mapped to other gestures, interactions, or commands, for example, based on a gesture vocabulary that specifies the mapping of gestures to commands).

The composite bioelectrodes disclosed herein may be implemented into, conformed to, and/or suitably shaped to fit a variety of wearable devices. In some examples, the terms “wearable” and “wearable device” may refer to any type or form of computing device that is worn by a user of an artificial-reality system and/or visual display system as part of an article of clothing, an accessory, and/or an implant. In one example, a wearable device may include and/or represent a wristband secured to and/or worn by the wrist of a user. Additional examples of wearable devices include, without limitation, armbands, pendants, bracelets, rings, jewelry, anklebands, clothing, electronic textiles, shoes, clips, headsets, headbands, head-mounted displays, gloves, glasses, variations or combinations of one or more of the same, and/or any other suitable wearable devices.

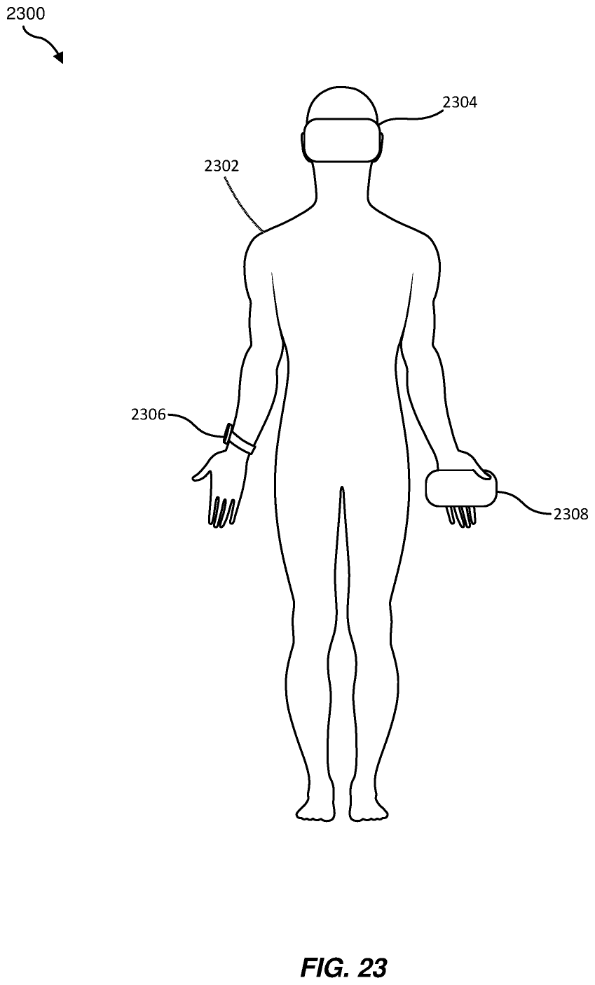

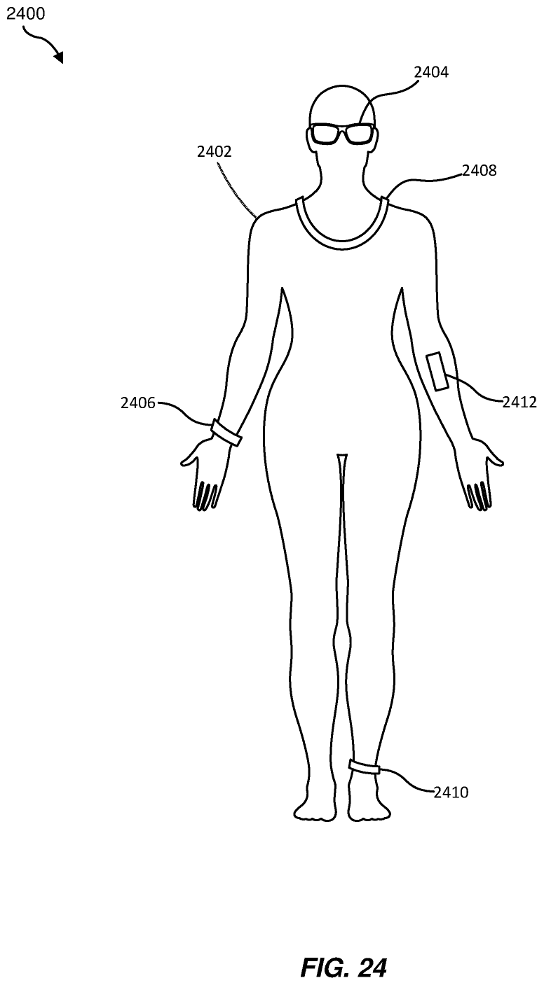

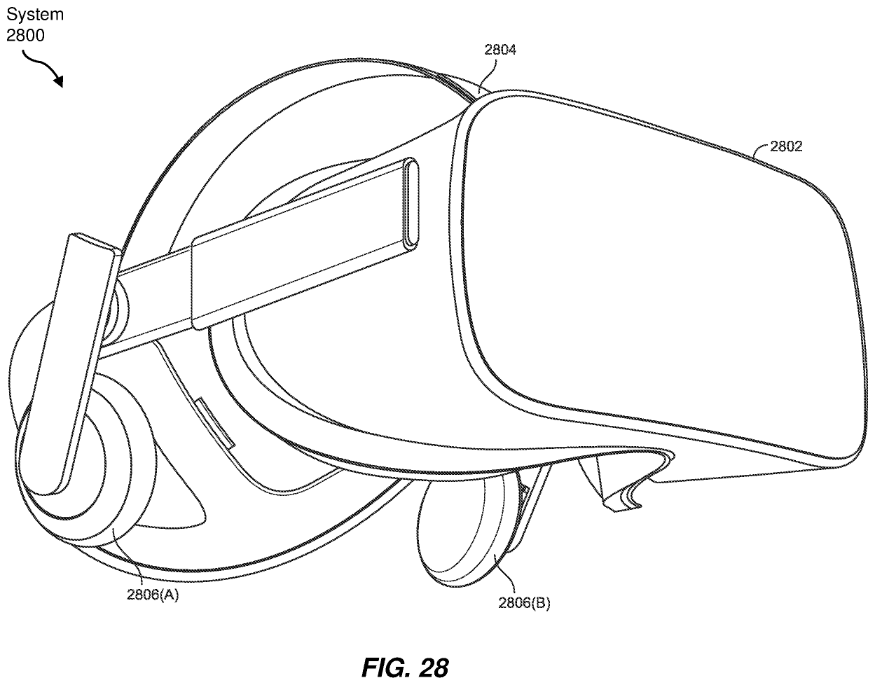

Composite bioelectrode(s) 110 and/or composite bioelectrode array 1600 may be implemented into one or more of the devices in example systems 2300 and 2400 shown in FIGS. 23 and 24. As shown in FIG. 23, system 2300 may include a user 2302 and computing devices that are worn or held by user 2302. For example, FIG. 23 illustrates a head-mounted display system 2304, such as head-mounted display system 2800 shown in FIG. 28, worn on the head of user 2302, a smart watch 2306 worn on a wrist of user 2302, and a smart phone 2308 held in a hand of user 2302. As shown in FIG. 24, system 2400 may include a user 2402 and various computing devices that are worn or held by user 2402. For example, FIG. 24 illustrates a head-mounted display device 2404, such as head-mounted display device 2700 illustrated in FIG. 27, worn on the head of user 2402, an electronic device 2406 worn on a wrist of user 2402, an electronic device 2408 worn about neck region of user 2402, an electronic device 2410 worn on an ankle of user 2402, and a flexible electronic device 2412 worn on a forearm of user 2402. In some examples, one or more of the devices shown in FIGS. 23 and 24 may be shaped to conform to a corresponding portion of the wearers' bodies.

The various devices, systems, and methods described herein may involve the use of a wearable device capable of detecting and/or sensing neuromuscular signals traversing through a user's body. For example, a user may wear a smart wristband with multiple surface electromyography (EMG) sensors that detect and/or sense neuromuscular signals traversing the user's arm, wrist, and/or hand. In this example, the smart wristband may be communicatively coupled to a nearby computing device. In response to certain neuromuscular signals detected via the user's body, the smart wristband may direct the computing device to perform one or more actions that account for those neuromuscular signals.

Accordingly, the smart wristband may enable the user to engage with interactive media presented and/or displayed on the computing device in less restrictive ways than traditional HCIs. The smart wristband may be used to control certain elements of interactive media based at least in part on EMG signals that correlate to predefined states of one or more body parts of the user. The smart wristband may enable the user to direct the computing device to perform certain interactive tasks. Examples of such interactive tasks include, without limitation, map navigation, page browsing, gaming controls, flight controls, interactions with graphical objects presented on a display, cursor control, link and/or button selection, combinations of one or more of the same, and/or any other suitable interactive tasks.

In some implementations, a wearable device may be used to transition between different mappings of body part states and responsive actions. For example, the wearable device may detect and/or sense certain neuromuscular signals traversing a user's body. In this example, those neuromuscular signals may correspond to and/or represent a specific state of one or more of the user's body parts. As a result, the wearable device may be able to detect and/or sense one or more positions, movements, forces, contractions, poses, and/or gestures made by those body parts of the user. One mapping may cause the wearable device and/or the target computing device to perform a certain action in response to the detection of a specific state of those body parts. However, another mapping may cause the wearable device and/or the target computing device to perform a different action in response to the detection of the same state of those body parts. The wearable device may enable the user to transition between those mappings via neuromuscular signals.

Embodiments of the present disclosure may include or be implemented in conjunction with various types of artificial-reality systems. Artificial reality is a form of reality that has been adjusted in some manner before presentation to a user, which may include, for example, a virtual reality, an augmented reality, a mixed reality, a hybrid reality, or some combination and/or derivative thereof. Artificial-reality content may include completely computer-generated content or computer-generated content combined with captured (e.g., real-world) content. The artificial-reality content may include video, audio, haptic feedback, or some combination thereof, any of which may be presented in a single channel or in multiple channels (such as stereo video that produces a three-dimensional (3D) effect to the viewer). Additionally, in some embodiments, artificial reality may also be associated with applications, products, accessories, services, or some combination thereof, that are used to, for example, create content in an artificial reality and/or are otherwise used in (e.g., to perform activities in) an artificial reality.

Artificial-reality systems may be implemented in a variety of different form factors and configurations. Some artificial-reality systems may be designed to work without near-eye displays (NEDs), an example of which is augmented-reality system 2500 in FIG. 25. Other artificial-reality systems may include an NED that also provides visibility into the real world (e.g., augmented-reality system 2600 in FIG. 26) or that visually immerses a user in an artificial reality (e.g., virtual-reality system 2800 in FIG. 28). While some artificial-reality devices may be self-contained systems, other artificial-reality devices may communicate and/or coordinate with external devices to provide an artificial-reality experience to a user. Examples of such external devices include handheld controllers, mobile devices, desktop computers, devices worn by a user, devices worn by one or more other users, and/or any other suitable external system.

Turning to FIG. 25, augmented-reality system 2500 generally represents a wearable device dimensioned to fit about a body part (e.g., a head) of a user. As shown in FIG. 25, system 2500 may include a frame 2502 and a camera assembly 2504 that is coupled to frame 2502 and configured to gather information about a local environment by observing the local environment. Augmented-reality system 2500 may also include one or more audio devices, such as output audio transducers 2508(A) and 2508(B) and input audio transducers 2510. Output audio transducers 2508(A) and 2508(B) may provide audio feedback and/or content to a user, and input audio transducers 2510 may capture audio in a user's environment.

As shown, augmented-reality system 2500 may not necessarily include an NED positioned in front of a user's eyes. Augmented-reality systems without NEDs may take a variety of forms, such as head bands, hats, hair bands, belts, watches, wristbands, ankle bands, rings, neckbands, necklaces, chest bands, eyewear frames, and/or any other suitable type or form of apparatus. While augmented-reality system 2500 may not include an NED, augmented-reality system 2500 may include other types of screens or visual feedback devices (e.g., a display screen integrated into a side of frame 2502).

The embodiments discussed in this disclosure may also be implemented in augmented-reality systems that include one or more NEDs. For example, as shown in FIG. 26, augmented-reality system 2600 may include an eyewear device 2602 with a frame 2610 configured to hold a left display device 2615(A) and a right display device 2615(B) in front of a user's eyes. Display devices 2615(A) and 2615(B) may act together or independently to present an image or series of images to a user. While augmented-reality system 2600 includes two displays, embodiments of this disclosure may be implemented in augmented-reality systems with a single NED or more than two NEDs.

In some embodiments, augmented-reality system 2600 may include one or more sensors, such as sensor 2640. Sensor 2640 may generate measurement signals in response to motion of augmented-reality system 2600 and may be located on substantially any portion of frame 2610. Sensor 2640 may represent a position sensor, an inertial measurement unit (IMU), a depth camera assembly, or any combination thereof. In some embodiments, augmented-reality system 2600 may or may not include sensor 2640 or may include more than one sensor. In embodiments in which sensor 2640 includes an IMU, the IMU may generate calibration data based on measurement signals from sensor 2640. Examples of sensor 2640 may include, without limitation, accelerometers, gyroscopes, magnetometers, other suitable types of sensors that detect motion, sensors used for error correction of the IMU, or some combination thereof. Augmented-reality system 2600 may also include a microphone array with a plurality of acoustic transducers 2620(A)-2620(J), referred to collectively as acoustic transducers 2620. Acoustic transducers 2620 may be transducers that detect air pressure variations induced by sound waves. Each acoustic transducer 2620 may be configured to detect sound and convert the detected sound into an electronic format (e.g., an analog or digital format). The microphone array in FIG. 2 may include, for example, ten acoustic transducers: 2620(A) and 2620(B), which may be designed to be placed inside a corresponding ear of the user, acoustic transducers 2620(C), 2620(D), 2620(E), 2620(F), 2620(G), and 2620(H), which may be positioned at various locations on frame 2610, and/or acoustic transducers 2620(1) and 2620(J), which may be positioned on a corresponding neckband 2605.

In some embodiments, one or more of acoustic transducers 2620(A)-(F) may be used as output transducers (e.g., speakers). For example, acoustic transducers 2620(A) and/or 2620(B) may be earbuds or any other suitable type of headphone or speaker.

The configuration of acoustic transducers 2620 of the microphone array may vary. While augmented-reality system 2600 is shown in FIG. 26 as having ten acoustic transducers 2620, the number of acoustic transducers 2620 may be greater or less than ten. In some embodiments, using higher numbers of acoustic transducers 2620 may increase the amount of audio information collected and/or the sensitivity and accuracy of the audio information. In contrast, using a lower number of acoustic transducers 2620 may decrease the computing power required by an associated controller 2650 to process the collected audio information. In addition, the position of each acoustic transducer 2620 of the microphone array may vary. For example, the position of an acoustic transducer 2620 may include a defined position on the user, a defined coordinate on frame 2610, an orientation associated with each acoustic transducer 2620, or some combination thereof.

Acoustic transducers 2620(A) and 2620(B) may be positioned on different parts of the user's ear, such as behind the pinna or within the auricle or fossa. Or, there may be additional acoustic transducers 2620 on or surrounding the ear in addition to acoustic transducers 2620 inside the ear canal. Having an acoustic transducer 2620 positioned next to an ear canal of a user may enable the microphone array to collect information on how sounds arrive at the ear canal. By positioning at least two of acoustic transducers 2620 on either side of a user's head (e.g., as binaural microphones), augmented-reality device 2600 may simulate binaural hearing and capture a 3D stereo sound field around about a user's head. In some embodiments, acoustic transducers 2620(A) and 2620(B) may be connected to augmented-reality system 2600 via a wired connection 2630, and in other embodiments, acoustic transducers 2620(A) and 2620(B) may be connected to augmented-reality system 2600 via a wireless connection (e.g., a Bluetooth connection). In still other embodiments, acoustic transducers 2620(A) and 2620(B) may not be used at all in conjunction with augmented-reality system 2600.

Acoustic transducers 2620 on frame 2610 may be positioned along the length of the temples, across the bridge, above or below display devices 2615(A) and 2615(B), or some combination thereof. Acoustic transducers 2620 may be oriented such that the microphone array is able to detect sounds in a wide range of directions surrounding the user wearing the augmented-reality system 2600. In some embodiments, an optimization process may be performed during manufacturing of augmented-reality system 2600 to determine relative positioning of each acoustic transducer 2620 in the microphone array.

In some examples, augmented-reality system 2600 may include or be connected to an external device (e.g., a paired device), such as neckband 2605. Neckband 2605 generally represents any type or form of paired device. Thus, the following discussion of neckband 2605 may also apply to various other paired devices, such as charging cases, smart watches, smart phones, wristbands, other wearable devices, hand-held controllers, tablet computers, laptop computers and other external compute devices, etc.

As shown, neckband 2605 may be coupled to eyewear device 2602 via one or more connectors. The connectors may be wired or wireless and may include electrical and/or non-electrical (e.g., structural) components. In some cases, eyewear device 2602 and neckband 2605 may operate independently without any wired or wireless connection between them. While FIG. 26 illustrates the components of eyewear device 2602 and neckband 2605 in example locations on eyewear device 2602 and neckband 2605, the components may be located elsewhere and/or distributed differently on eyewear device 2602 and/or neckband 2605. In some embodiments, the components of eyewear device 2602 and neckband 2605 may be located on one or more additional peripheral devices paired with eyewear device 2602, neckband 2605, or some combination thereof.

Pairing external devices, such as neckband 2605, with augmented-reality eyewear devices may enable the eyewear devices to achieve the form factor of a pair of glasses while still providing sufficient battery and computation power for expanded capabilities. Some or all of the battery power, computational resources, and/or additional features of augmented-reality system 2600 may be provided by a paired device or shared between a paired device and an eyewear device, thus reducing the weight, heat profile, and form factor of the eyewear device overall while still retaining desired functionality. For example, neckband 2605 may allow components that would otherwise be included on an eyewear device to be included in neckband 2605 since users may tolerate a heavier weight load on their shoulders than they would tolerate on their heads. Neckband 2605 may also have a larger surface area over which to diffuse and disperse heat to the ambient environment. Thus, neckband 2605 may allow for greater battery and computation capacity than might otherwise have been possible on a stand-alone eyewear device. Since weight carried in neckband 2605 may be less invasive to a user than weight carried in eyewear device 2602, a user may tolerate wearing a lighter eyewear device and carrying or wearing the paired device for greater lengths of time than a user would tolerate wearing a heavy standalone eyewear device, thereby enabling users to more fully incorporate artificial-reality environments into their day-to-day activities.

Neckband 2605 may be communicatively coupled with eyewear device 2602 and/or to other devices. These other devices may provide certain functions (e.g., tracking, localizing, depth mapping, processing, storage, etc.) to augmented-reality system 2600. In the embodiment of FIG. 26, neckband 2605 may include two acoustic transducers (e.g., 2620(1) and 2620(J)) that are part of the microphone array (or potentially form their own microphone subarray). Neckband 2605 may also include a controller 2625 and a power source 2635.

Acoustic transducers 2620(1) and 2620(J) of neckband 2605 may be configured to detect sound and convert the detected sound into an electronic format (analog or digital). In the embodiment of FIG. 26, acoustic transducers 2620(1) and 2620(J) may be positioned on neckband 2605, thereby increasing the distance between the neckband acoustic transducers 2620(1) and 2620(J) and other acoustic transducers 2620 positioned on eyewear device 2602. In some cases, increasing the distance between acoustic transducers 2620 of the microphone array may improve the accuracy of beamforming performed via the microphone array. For example, if a sound is detected by acoustic transducers 2620(C) and 2620(D) and the distance between acoustic transducers 2620(C) and 2620(D) is greater than, e.g., the distance between acoustic transducers 2620(D) and 2620(E), the determined source location of the detected sound may be more accurate than if the sound had been detected by acoustic transducers 2620(D) and 2620(E).

Controller 2625 of neckband 2605 may process information generated by the sensors on neckband 2605 and/or augmented-reality system 2600. For example, controller 2625 may process information from the microphone array that describes sounds detected by the microphone array. For each detected sound, controller 2625 may perform a direction-of-arrival (DOA) estimation to estimate a direction from which the detected sound arrived at the microphone array. As the microphone array detects sounds, controller 2625 may populate an audio data set with the information. In embodiments in which augmented-reality system 2600 includes an inertial measurement unit, controller 2625 may compute all inertial and spatial calculations from the IMU located on eyewear device 2602. A connector may convey information between augmented-reality system 2600 and neckband 2605 and between augmented-reality system 2600 and controller 2625. The information may be in the form of optical data, electrical data, wireless data, or any other transmittable data form. Moving the processing of information generated by augmented-reality system 2600 to neckband 2605 may reduce weight and heat in eyewear device 2602, making it more comfortable to the user.

Power source 2635 in neckband 2605 may provide power to eyewear device 2602 and/or to neckband 2605. Power source 2635 may include, without limitation, lithium ion batteries, lithium-polymer batteries, primary lithium batteries, alkaline batteries, or any other form of power storage. In some cases, power source 2635 may be a wired power source. Including power source 2635 on neckband 2605 instead of on eyewear device 2602 may help better distribute the weight and heat generated by power source 2635.

FIG. 27 is an illustration of a head-mounted display device 2700 according to some embodiments. The depicted embodiment includes a right near-eye display 2702A and a left near-eye display 2702B, which are collectively referred to as near-eye displays 2702. Near-eye displays 2702 may be transparent or semi-transparent lenses that include or utilize a display system (e.g., a projection display system) to present media to a user. Examples of media presented by near-eye displays 2702 include one or more images, a series of images (e.g., a video), audio, or some combination thereof. Near-eye displays 2702 may be configured to operate as an augmented-reality near-eye display, such that a user can see media projected by near-eye displays 2702 and see the real-world environment through near-eye displays 2702. However, in some embodiments, near-eye displays 2702 may be modified to also operate as virtual-reality near-eye displays, mixed-reality near-eye displays, or some combination thereof. Accordingly, in some embodiments, near-eye displays 2702 may augment views of a physical, real-world environment with computer-generated elements (e.g., images, video, sound, etc.).

As shown in FIG. 27, head-mounted display device 2700 may include a support or frame 2704 that secures near-eye displays 2702 in place on the head of a user, in embodiments in which near-eye displays 2702 includes separate left and right displays. In some embodiments, frame 2704 may be a frame of eye-wear glasses. Frame 2704 may include temples 2706 configured to rest on the top of and/or behind a user's ears, a bridge 2708 configured to rest on the top on the bridge of the user's nose, and rims 2710 sized and configured to rest on or against the user's cheeks. In various embodiments, any or all of the components of frame 2704 may include or integrate the curved batteries disclosed herein. Although not illustrated in FIG. 27, in some embodiments, head-mounted display device 2700 may include nose pads for resting on the bridge of the user's nose. Head-mounted-display device 2700 may additionally or alternatively include various other features and/or components, including, for example, directional speakers to provide audio to a user, bone conduction transducers for providing sound signals to a user via vibrational bone conduction in an auditory region of the user's head, tracking and/or recording cameras, passive and/or active front and/or rear facing cameras to capture images from the user's environment, eye tracking cameras, ambient light, night vision, and/or thermal imaging sensors, multimode connectivity antennas for wireless communication, audio microphones for capturing sound in the user's environment, lights for illuminating a user's environment, inertial, haptic, environmental, and/or health monitoring sensors, and/or any other suitable components, without limitation.

As noted, some artificial-reality systems may, instead of blending an artificial reality with actual reality, substantially replace one or more of a user's sensory perceptions of the real world with a virtual experience. One example of this type of system is a head-worn display system, such as virtual-reality system 2800 in FIG. 28, that mostly or completely covers a user's field of view. Virtual-reality system 2800 may include a front rigid body 2802 and a band 2804 shaped to fit around a user's head. Virtual-reality system 2800 may also include output audio transducers 2806(A) and 2806(B). Furthermore, while not shown in FIG. 28, front rigid body 2802 may include one or more electronic elements, including one or more electronic displays, one or more inertial measurement units (IMUS), one or more tracking emitters or detectors, and/or any other suitable device or system for creating an artificial reality experience.

Artificial-reality systems may include a variety of types of visual feedback mechanisms. For example, display devices in augmented-reality system 2600 and/or virtual-reality system 2800 may include one or more liquid crystal displays (LCDs), light emitting diode (LED) displays, organic LED (OLED) displays digital light project (DLP) micro-displays, liquid crystal on silicon (LCoS) micro-displays, and/or any other suitable type of display screen. Artificial-reality systems may include a single display screen for both eyes or may provide a display screen for each eye, which may allow for additional flexibility for varifocal adjustments or for correcting a user's refractive error. Some artificial-reality systems may also include optical subsystems having one or more lenses (e.g., conventional concave or convex lenses, Fresnel lenses, adjustable liquid lenses, etc.) through which a user may view a display screen. These optical subsystems may serve a variety of purposes, including to collimate (e.g., make an object appear at a greater distance than its physical distance), to magnify (e.g., make an object appear larger than its actual size), and/or to relay (to, e.g., the viewer's eyes) light. These optical subsystems may be used in a non-pupil-forming architecture (such as a single lens configuration that directly collimates light but results in so-called pincushion distortion) and/or a pupil-forming architecture (such as a multi-lens configuration that produces so-called barrel distortion to nullify pincushion distortion).

In addition to or instead of using display screens, some artificial-reality systems may include one or more projection systems. For example, display devices in augmented-reality system 2600 and/or virtual-reality system 2800 may include micro-LED projectors that project light (using, e.g., a waveguide) into display devices, such as clear combiner lenses that allow ambient light to pass through. The display devices may refract the projected light toward a user's pupil and may enable a user to simultaneously view both artificial-reality content and the real world. The display devices may accomplish this using any of a variety of different optical components, including waveguides components (e.g., holographic, planar, diffractive, polarized, and/or reflective waveguide elements), light-manipulation surfaces and elements (such as diffractive, reflective, and refractive elements and gratings), coupling elements, etc. Artificial-reality systems may also be configured with any other suitable type or form of image projection system, such as retinal projectors used in virtual retina displays.

Artificial-reality systems may also include various types of computer vision components and subsystems. For example, augmented-reality system 2500, augmented-reality system 2600, and/or virtual-reality system 2800 may include one or more optical sensors, such as two-dimensional (2D) or 3D cameras, time-of-flight depth sensors, single-beam or sweeping laser rangefinders, 3D LiDAR sensors, and/or any other suitable type or form of optical sensor. An artificial-reality system may process data from one or more of these sensors to identify a location of a user, to map the real world, to provide a user with context about real-world surroundings, and/or to perform a variety of other functions.

Artificial-reality systems may also include one or more input and/or output audio transducers. In the examples shown in FIGS. 25 and 28, output audio transducers 2508(A), 2508(B), 2806(A), and 2806(B) may include voice coil speakers, ribbon speakers, electrostatic speakers, piezoelectric speakers, bone conduction transducers, cartilage conduction transducers, and/or any other suitable type or form of audio transducer. Similarly, input audio transducers 2510 may include condenser microphones, dynamic microphones, ribbon microphones, and/or any other type or form of input transducer. In some embodiments, a single transducer may be used for both audio input and audio output.

While not shown in FIGS. 25-28, artificial-reality systems may include tactile (i.e., haptic) feedback systems, which may be incorporated into headwear, gloves, body suits, handheld controllers, environmental devices (e.g., chairs, floormats, etc.), and/or any other type of device or system. Haptic feedback systems may provide various types of cutaneous feedback, including vibration, force, traction, texture, and/or temperature. Haptic feedback systems may also provide various types of kinesthetic feedback, such as motion and compliance. Haptic feedback may be implemented using motors, piezoelectric actuators, fluidic systems, and/or a variety of other types of feedback mechanisms. Haptic feedback systems may be implemented independent of other artificial-reality devices, within other artificial-reality devices, and/or in conjunction with other artificial-reality devices.

By providing haptic sensations, audible content, and/or visual content, artificial-reality systems may create an entire virtual experience or enhance a user's real-world experience in a variety of contexts and environments. For instance, artificial-reality systems may assist or extend a user's perception, memory, or cognition within a particular environment. Some systems may enhance a user's interactions with other people in the real world or may enable more immersive interactions with other people in a virtual world. Artificial-reality systems may also be used for educational purposes (e.g., for teaching or training in schools, hospitals, government organizations, military organizations, business enterprises, etc.), entertainment purposes (e.g., for playing video games, listening to music, watching video content, etc.), and/or for accessibility purposes (e.g., as hearing aids, visuals aids, etc.). The embodiments disclosed herein may enable or enhance a user's artificial-reality experience in one or more of these contexts and environments and/or in other contexts and environments.

As noted, artificial-reality systems 2500, 2600, and 2800 may be used with a variety of other types of devices to provide a more compelling artificial-reality experience. These devices may be haptic interfaces with transducers that provide haptic feedback and/or that collect haptic information about a user's interaction with an environment. The artificial-reality systems disclosed herein may include various types of haptic interfaces that detect or convey various types of haptic information, including tactile feedback (e.g., feedback that a user detects via nerves in the skin, which may also be referred to as cutaneous feedback) and/or kinesthetic feedback (e.g., feedback that a user detects via receptors located in muscles, joints, and/or tendons).

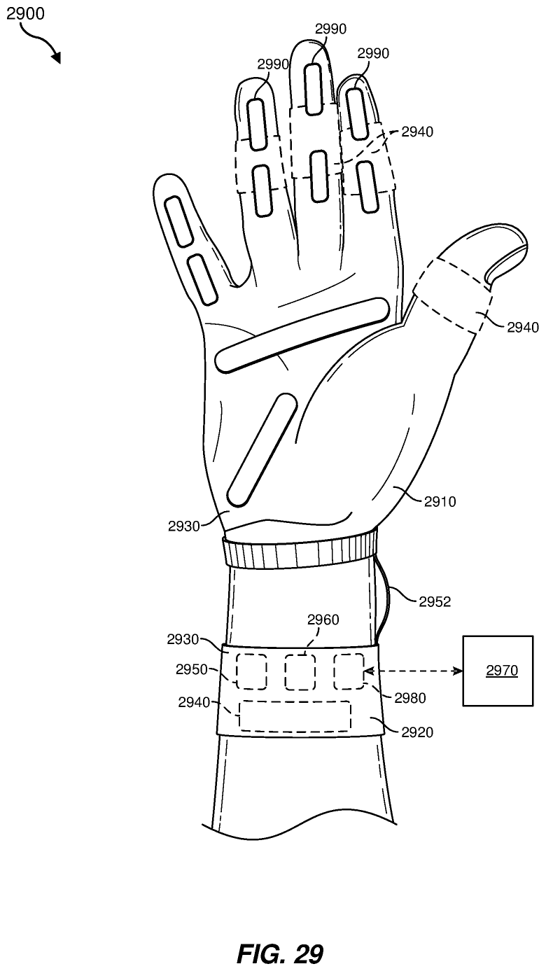

Haptic feedback may be provided by interfaces positioned within a user's environment (e.g., chairs, tables, floors, etc.) and/or interfaces on articles that may be worn or carried by a user (e.g., gloves, wristbands, etc.). As an example, FIG. 29 illustrates a vibrotactile system 2900 in the form of a wearable glove (haptic device 2910) and wristband (haptic device 2920). Haptic device 2910 and haptic device 2920 are shown as examples of wearable devices that include a flexible, wearable textile material 2930 that is shaped and configured for positioning against a user's hand and wrist, respectively. This disclosure also includes vibrotactile systems that may be shaped and configured for positioning against other human body parts, such as a finger, an arm, a head, a torso, a foot, or a leg. By way of example and not limitation, vibrotactile systems according to various embodiments of the present disclosure may also be in the form of a glove, a headband, an armband, a sleeve, a head covering, a sock, a shirt, or pants, among other possibilities. In some examples, the term “textile” may include any flexible, wearable material, including woven fabric, non-woven fabric, leather, cloth, a flexible polymer material, composite materials, etc.

One or more vibrotactile devices 2940 may be positioned at least partially within one or more corresponding pockets formed in textile material 2930 of vibrotactile system 2900. Vibrotactile devices 2940 may be positioned in locations to provide a vibrating sensation (e.g., haptic feedback) to a user of vibrotactile system 2900. For example, vibrotactile devices 2940 may be positioned against the user's finger(s), thumb, or wrist, as shown in FIG. 29. Vibrotactile devices 2940 may, in some examples, be sufficiently flexible to conform to or bend with the user's corresponding body part(s).

A power source 2950 (e.g., a battery) for applying a voltage to the vibrotactile devices 2940 for activation thereof may be electrically coupled to vibrotactile devices 2940, such as via conductive wiring 2952. In some examples, each of vibrotactile devices 2940 may be independently electrically coupled to power source 2950 for individual activation. In some embodiments, a processor 2960 may be operatively coupled to power source 2950 and configured (e.g., programmed) to control activation of vibrotactile devices 2940.

Vibrotactile system 2900 may be implemented in a variety of ways. In some examples, vibrotactile system 2900 may be a standalone system with integral subsystems and components for operation independent of other devices and systems. As another example, vibrotactile system 2900 may be configured for interaction with another device or system 2970. For example, vibrotactile system 2900 may, in some examples, include a communications interface 2980 for receiving and/or sending signals to the other device or system 2970. The other device or system 2970 may be a mobile device, a gaming console, an artificial-reality (e.g., virtual-reality, augmented-reality, mixed-reality) device, a personal computer, a tablet computer, a network device (e.g., a modem, a router, etc.), a handheld controller, etc. Communications interface 2980 may enable communications between vibrotactile system 2900 and the other device or system 2970 via a wireless (e.g., Wi-Fi, Bluetooth, cellular, radio, etc.) link or a wired link. If present, communications interface 2980 may be in communication with processor 2960, such as to provide a signal to processor 2960 to activate or deactivate one or more of the vibrotactile devices 2940.

Vibrotactile system 2900 may optionally include other subsystems and components, such as touch-sensitive pads 2990, pressure sensors, motion sensors, position sensors, lighting elements, and/or user interface elements (e.g., an on/off button, a vibration control element, etc.). During use, vibrotactile devices 2940 may be configured to be activated for a variety of different reasons, such as in response to the user's interaction with user interface elements, a signal from the motion or position sensors, a signal from the touch-sensitive pads 2990, a signal from the pressure sensors, a signal from the other device or system 2970, etc.

Although power source 2950, processor 2960, and communications interface 2980 are illustrated in FIG. 29 as being positioned in haptic device 2920, the present disclosure is not so limited. For example, one or more of power source 2950, processor 2960, or communications interface 2980 may be positioned within haptic device 2910 or within another wearable textile.

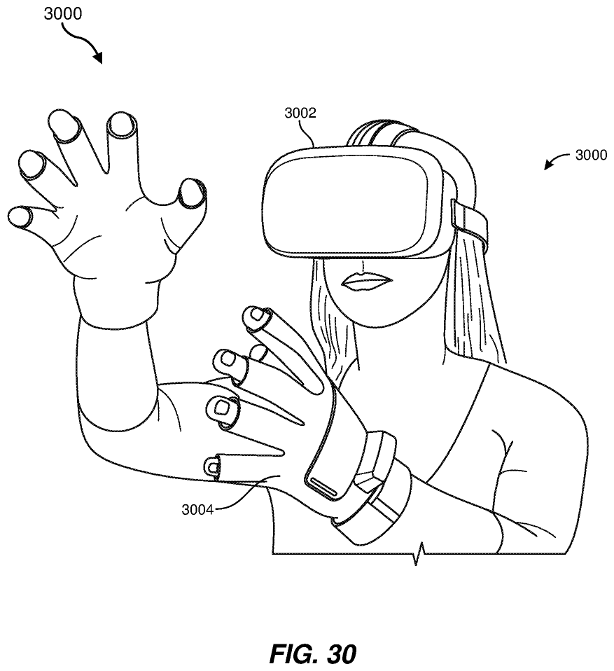

Haptic wearables, such as those shown in and described in connection with FIG. 29, may be implemented in a variety of types of artificial-reality systems and environments. FIG. 30 shows an example artificial-reality environment 3000 including one head-mounted virtual-reality display and two haptic devices (i.e., gloves), and in other embodiments any number and/or combination of these components and other components may be included in an artificial-reality system. For example, in some embodiments there may be multiple head-mounted displays each having an associated haptic device, with each head-mounted display and each haptic device communicating with the same console, portable computing device, or other computing system.

Head-mounted display 3002 generally represents any type or form of virtual-reality system, such as virtual-reality system 2800 in FIG. 28. Haptic device 3004 generally represents any type or form of wearable device, worn by a user of an artificial-reality system, that provides haptic feedback to the user to give the user the perception that he or she is physically engaging with a virtual object. In some embodiments, haptic device 3004 may provide haptic feedback by applying vibration, motion, and/or force to the user. For example, haptic device 3004 may limit or augment a user's movement. To give a specific example, haptic device 3004 may limit a user's hand from moving forward so that the user has the perception that his or her hand has come in physical contact with a virtual wall. In this specific example, one or more actuators within the haptic advice may achieve the physical-movement restriction by pumping fluid into an inflatable bladder of the haptic device. In some examples, a user may also use haptic device 3004 to send action requests to a console. Examples of action requests include, without limitation, requests to start an application and/or end the application and/or requests to perform a particular action within the application.

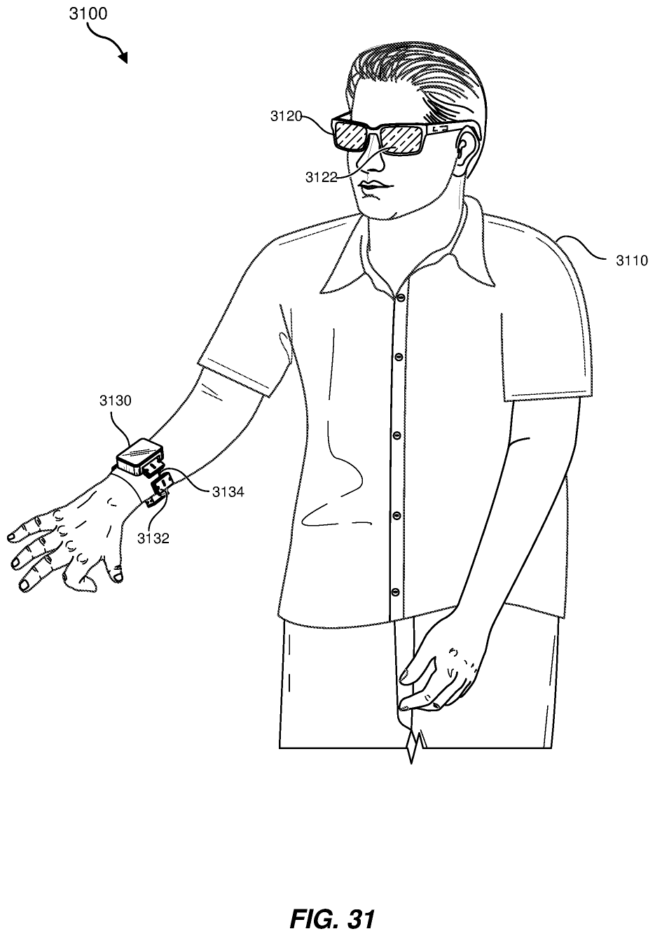

While haptic interfaces may be used with virtual-reality systems, as shown in FIG. 30, haptic interfaces may also be used with augmented-reality systems, as shown in FIG. 31. FIG. 31 is a perspective view of a user 3110 interacting with an augmented-reality system 3100. In this example, user 3110 may wear a pair of augmented-reality glasses 3120 that may have one or more displays 3122 and that are paired with a haptic device 3130. In this example, haptic device 3130 may be a wristband that includes a plurality of band elements 3132 and a tensioning mechanism 3134 that connects band elements 3132 to one another.

One or more of band elements 3132 may include any type or form of actuator suitable for providing haptic feedback. For example, one or more of band elements 3132 may be configured to provide one or more of various types of cutaneous feedback, including vibration, force, traction, texture, and/or temperature. To provide such feedback, band elements 3132 may include one or more of various types of actuators. In one example, each of band elements 3132 may include a vibrotactor (e.g., a vibrotactile actuator) configured to vibrate in unison or independently to provide one or more of various types of haptic sensations to a user. Alternatively, only a single band element or a subset of band elements may include vibrotactors.

Haptic devices 2910, 2920, 3004, and 3130 may include any suitable number and/or type of haptic transducer, sensor, and/or feedback mechanism. For example, haptic devices 2910, 2920, 3004, and 3130 may include one or more mechanical transducers, piezoelectric transducers, and/or fluidic transducers. Haptic devices 2910, 2920, 3004, and 3130 may also include various combinations of different types and forms of transducers that work together or independently to enhance a user's artificial-reality experience. In one example, each of band elements 3132 of haptic device 3130 may include a vibrotactor (e.g., a vibrotactile actuator) configured to vibrate in unison or independently to provide one or more of various types of haptic sensations to a user.

### Example Embodiments

Example 1: A computing device including (1) biosignal-acquiring circuitry that captures a biosignal from a user's body and (2) one or more composite bioelectrodes, communicatively coupled to the biosignal-acquiring circuitry, that convey the biosignal from the user's body to the biosignal-acquiring circuitry. The one or more composite bioelectrodes may include (a) a circuitry-interfacing side with a mechanical or electrical property having a first predetermined configuration and (b) a user-interfacing side with the mechanical or electrical property having a second predetermined configuration.

Example 2: The computing device of Example 1, wherein the first predetermined configuration matches an associated configuration of the mechanical or electrical property of the biosignal-acquiring circuitry.

Example 3: The computing device of any of Examples 1-2, wherein the second predetermined configuration matches an associated configuration of the mechanical or electrical property of the user's body.

Example 4: The computing device of any of Examples 1-3, wherein the mechanical or electrical property is one of a surface texture or a geometry.

Example 5: The computing device of any of Examples 1-4, wherein the mechanical or electrical property is a stiffness.

Example 6: The computing device of any of Examples 1-5, wherein the mechanical or electrical property is an impedance.

Example 7: The computing device of any of Examples 1-6, wherein the mechanical or electrical property is a softness.

Example 8: The computing device of any of Examples 1-7, wherein the second predetermined configuration minimizes an effect of pressure on a conductivity of the one or more composite bioelectrodes or maximizes comfort for the user.

Example 9: The computing device of any of Examples 1-8, wherein the one or more composite bioelectrodes are integrated into a wristband of the computing device.

10: The computing device of any of Examples 1-9, wherein the one or more composite bioelectrodes form a monolithic array and each of the one or more composite bioelectrodes is electrically isolated and anisotropically conductive.

Example 11: A composite bioelectrode for conducting a biosignal from a user's body to biosignal-acquiring circuitry, the composite bioelectrode including (1) a circuitry-interfacing portion that interfaces with the biosignal-acquiring circuitry, the circuitry-interfacing portion having a mechanical or electrical property equal to a first predetermined value and (2) a user-interfacing portion that interfaces with the user's body, the user-interfacing portion having the mechanical or electrical property equal to a second predetermined value.

Example 12: The composite bioelectrode of Example 11, further comprising one or more intermediate portions between the circuitry-interfacing portion and the user-interfacing portion.

Example 13: The composite bioelectrode of Example 12, wherein values of the mechanical or electrical property of the one or more intermediate portions vary continuously between the first predetermined value and the second predetermined value.

Example 14: The composite bioelectrode of Example 12, wherein values of the mechanical or electrical property of the one or more intermediate portions vary discontinuously between the first predetermined value and the second predetermined value.

Example 15: The composite bioelectrode of Example 12, wherein values of the mechanical or electrical property of the one or more intermediate portions monotonically increase from the first predetermined value and the second predetermined value.

Example 16: The composite bioelectrode of Example 12, wherein values of the mechanical or electrical property of the one or more intermediate portions monotonically decrease from the first predetermined value and the second predetermined value.

Example 17: A method of manufacturing a composite bioelectrode to conduct a biosignal from a user's body to biosignal-acquiring circuitry. The method may include (1) producing a circuitry-interfacing portion of the composite bioelectrode with a mechanical or electrical property equal to a first predetermined value and (2) producing a user-interfacing portion of the composite bioelectrode with the mechanical or electrical property equal to a second predetermined value.

Example 18: The method of Example 17, wherein the first predetermined value is tuned to match an associated value of the mechanical or electrical property of the biosignal-acquiring circuitry and the second predetermined value is tuned to match an associated value of the mechanical or electrical property of the user's body.

Example 19: The method of any of Examples 17 and 18, wherein the circuitry-interfacing portion and the user-interfacing portion of the composite bioelectrode are produced using one or more of injection molding, compression molding, doping, screen printing, photolithography, or three-dimensional printing.

Example 20: The method of any of Examples 17-19, wherein the composite bioelectrode is produced as part of a monolithic array of composite bioelectrodes.

The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or include additional steps in addition to those disclosed.

The preceding description has been provided to enable others skilled in the art to best utilize various aspects of the exemplary embodiments disclosed herein. This exemplary description is not intended to be exhaustive or to be limited to any precise form disclosed. Many modifications and variations are possible without departing from the spirit and scope of the present disclosure. The embodiments disclosed herein should be considered in all respects illustrative and not restrictive.

Unless otherwise noted, the terms “connected to” and “coupled to” (and their derivatives), as used in the specification, are to be construed as permitting both direct and indirect (i.e., via other elements or components) connection. In addition, the terms “a” or “an,” as used in the specification, are to be construed as meaning “at least one of.” Finally, for ease of use, the terms “including” and “having” (and their derivatives), as used in the specification, are interchangeable with and have the same meaning as the word “comprising.”

What is claimed is:

1. A computing device comprising:
biosignal-acquiring circuitry that captures a biosignal from a user's body; and
one or more composite bioelectrodes, communicatively coupled to the biosignal-acquiring circuitry, that convey the biosignal from the user's body to the biosignal-acquiring circuitry, the one or more composite bioelectrodes comprising:
a circuitry-interfacing side with a mechanical or electrical property having a first predetermined configuration; and
a user-interfacing side with the mechanical or electrical property having a second predetermined configuration.

2. The computing device of claim 1, wherein the first predetermined configuration matches an associated configuration of the mechanical or electrical property of the biosignal-acquiring circuitry.

3. The computing device of claim 1, wherein the second predetermined configuration matches an associated configuration of the mechanical or electrical property of the user's body.

4. The computing device of claim 1, wherein the mechanical or electrical property is one of a surface texture or a geometry.

5. The computing device of claim 1, wherein the mechanical or electrical property is a stiffness.

6. The computing device of claim 1, wherein the mechanical or electrical property is an impedance.

7. The computing device of claim 1, wherein the mechanical or electrical property is a softness.

8. The computing device of claim 7, wherein the second predetermined configuration:
minimizes an effect of pressure on a conductivity of the one or more composite bioelectrodes; or
maximizes comfort for the user.

9. The computing device of claim 1, wherein the one or more composite bioelectrodes are integrated into a wristband of the computing device.

10. The computing device of claim 1, wherein:
the one or more composite bioelectrodes form a monolithic array; and
each of the one or more composite bioelectrodes is electrically isolated and anisotropically conductive.

11. A composite bioelectrode for conducting a biosignal from a user's body to biosignal-acquiring circuitry, the composite bioelectrode comprising:
a circuitry-interfacing portion that interfaces with the biosignal-acquiring circuitry, the circuitry-interfacing portion having a mechanical or electrical property equal to a first predetermined value; and
a user-interfacing portion that interfaces with the user's body, the user-interfacing portion having the mechanical or electrical property equal to a second predetermined value.

12. The composite bioelectrode of claim 11, further comprising one or more intermediate portions between the circuitry-interfacing portion and the user-interfacing portion.

13. The composite bioelectrode of claim 12, wherein values of the mechanical or electrical property of the one or more intermediate portions vary continuously between the first predetermined value and the second predetermined value.

14. The composite bioelectrode of claim 12, wherein values of the mechanical or electrical property of the one or more intermediate portions vary discontinuously between the first predetermined value and the second predetermined value.

15. The composite bioelectrode of claim 12, wherein values of the mechanical or electrical property of the one or more intermediate portions monotonically increase from the first predetermined value and the second predetermined value.

16. The composite bioelectrode of claim 12, wherein values of the mechanical or electrical property of the one or more intermediate portions monotonically decrease from the first predetermined value and the second predetermined value.

17. A method of manufacturing a composite bioelectrode to conduct a biosignal from a user's body to biosignal-acquiring circuitry, the method comprising:
producing a circuitry-interfacing portion of the composite bioelectrode with a mechanical or electrical property equal to a first predetermined value; and
producing a user-interfacing portion of the composite bioelectrode with the mechanical or electrical property equal to a second predetermined value.

18. The method of claim 17, wherein:
the first predetermined value is tuned to match an associated value of the mechanical or electrical property of the biosignal-acquiring circuitry; and
the second predetermined value is tuned to match an associated value of the mechanical or electrical property of the user's body.

19. The method of claim 17, wherein the circuitry-interfacing portion and the user-interfacing portion of the composite bioelectrode are produced using one or more of:
injection molding;
compression molding;
doping;
screen printing;
photolithography; or
three-dimensional printing.

20. The method of claim 17, wherein the composite bioelectrode is produced as part of a monolithic array of composite bioelectrodes.
